(19) United States
(12) Patent Application Publication
(10) Pub. No.: US 2024/0044870 A1
(43) Pub. Date: Feb. 8, 2024

(54) METHODS AND REAGENTS FOR HIGH-THROUGHPUT DRUG SCREENING

(71) Applicants: SINGLERON (NANJING) BIOTECHNOLOGIES, LTD., Nanjing (CN); CHINESE PLA GENERAL HOSPITAL, Beijing (CN)

(72) Inventors: Kunlun HE, Beijing (CN); Zhilong JIA, Beijing (CN); Jue WU, Beijing (CN); Xinyu SONG, Beijing (CN); Yu LU, Nanjing (CN); Wenqi ZHU, Nanjing (CN)

(21) Appl. No.: 18/258,786

(22) PCT Filed: Dec. 22, 2021
(86) PCT No.: PCT/CN2021/140507

§ 371 Date: Jun. 21, 2023

(30) Foreign Application Priority Data
Dec. 22, 2020 (WO) PCT/CN2020/138349

(51) Int. Cl. G01N 33/50; C12Q 1/6869

(52) U.S. Cl. CPC G01N 33/5023; C12Q 1/6869; C12Q 2600/158; C12Q 2600/16

(57) ABSTRACT

The present disclosure provides methods, reagents, compositions, systems and kits for high-throughput drug screening by using barcode molecules. In some embodiments, the method uses one step RT-PCR to simplify the experimental procedure and avoid RNA contamination in the experimental process.

### CROSS-REFERENCE TO RELATED APPLICATIONS

This application is a U.S. national phase application under 35 U.S.C. § 371 of International Application No. PCT/CN2021/140507, filed on Dec. 22, 2021, which claims the benefit of priority to PCT Application No. PCT/CN2020/138349, filed on Dec. 22, 2020, the content of each of which is incorporated herein by reference in its entirety.

### BACKGROUND

### Field

The present disclosure relates generally to the field of molecular biology, specifically drug screening.

### Description of the Related Art

Drug screening plays a critical role in drug development and research. However, the complexity of biological system and substantially large number of candidate chemicals make this work time-consuming and cumbersome. Modern technologies such as yeast double hybridization, genetic engineering, high-throughput sequencing, and bioinformatics have been applied to speed up drug screening process.

High-throughput screening technologies with advanced molecular biology, cell biology, computer, and automatic control can be used in drug screening. In drug screening, at the cellular and molecular level, detection of different types of cellular signals associated with apoptosis, proliferation, or alterations of therapeutic targets can be used to screen for candidate drugs. For example, gene expression profiles detected can be applied to repurpose drugs, annotate the drug's function and illuminate the regulation of biological pathways.

RNA-seq is a useful tool to investigate drug effects using transcriptome changes as a proxy in high-throughput screening. It can simultaneously measure the expression levels of thousands of genes, providing insights into functional pathways and regulation of biological processes. In addition, RNA-Seq can provide rich information on selective splicing, allele-specific expression, unannotated exons, and new transcripts (gene or non-coding RNA), which facilitates the development of drug screening and pharmacological analysis. RNA-seq was used to demonstrate differential gene expression of human non-small cell lung cancer cell line H1299 treated with Polyphenon, revealing the mechanism of Polyphenon as an effective chemo-preventive reagent in the treatment of lung cancer. Transcriptional differences between resistant strains and different resistant strains were revealed and 228 differentially expressed genes were found in Candida albicans by RNA-seq, indicating that a new transcription factor CZF1 contributes to drug resistance and CZF1 encoding is the reason for drug resistance in the resistant strain.

While RNA-Seq is applicable to genome-wide analysis, there remains an urgent need to quantify expression of large sets of compounds under multiple experimental conditions. To address the limitation, multiple transcriptional profiling platforms have been developed. Targeted sequencing-based approaches, such as RASL-seq, can measure up to a few hundred specific genes or splicing events. RASL-seq is useful for studying genes of interest or genomic loci, where a focused panel of events can be assessed. The latest development is PLATE-Seq with regulatory network analysis. The proposed approach perform a strategy for barcoding and pooling cDNA libraries to substantially reduce the cost and complexity of multi-sample RNA-Seq and use network based algorithms for the highly reproducible inference of protein activity from low-depth RNA-Seq profile. Besides, the digital RNA with pertUrbation of Genes (DRUG-seq) is another powerful tool to assist novel compound mechanistic studies. The transcription of multiple compounds at different doses was detected and the compounds were grouped into functional clusters in term of mechanism of actions (MoAs) by DRUG-seq. With the feature of easier performance, higher throughput and unbiasedness, it has advantages over other technologies such as RASL-seq, PLATE-seq and L1000. However, these methods posts challenges in time and costs while screening large sets of compounds under multiple experimental conditions simultaneously. Thus, there remains a need for more efficient and cost-effective drug screening methods.

### SUMMARY

Provided herein include methods, reagents, compositions, systems and kits for high-throughput pharmaceutical screening.

Disclosed herein include methods of pharmaceutical screening. In some embodiments, the method comprises: introducing one or more cells into each partition of a plurality of partitions; subjecting the one or more cells in the partition to a pharmaceutical condition of a plurality of pharmaceutical conditions; introducing a plurality of barcode molecules into the partition, wherein the barcode molecules each comprises a partition barcode sequence and a molecular barcode sequence; barcoding a plurality of target nucleic acids associated with the one or more cells in the partition using the plurality of the barcode molecules to generate a plurality of barcoded nucleic acids; subjecting the plurality of barcoded nucleic acids, or products thereof, to sequencing to obtain sequences of the barcoded nucleic acids; determining a profile of the one or more cells from the sequences of the barcoded nucleic acids; and determining a difference in the profile of the cells subjected to two different pharmaceutical conditions of the plurality of conditions. In some embodiments, the method of pharmaceutical screening comprises: providing a plurality of partitions each comprising one or more cells, or lysates thereof, subjected to a pharmaceutical condition of a plurality of pharmaceutical conditions; barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence; subjecting the plurality of barcoded nucleic acids, or products thereof, to sequencing to obtain sequences of the barcoded nucleic acids; determining a profile of the one or more cells from the sequences of the barcoded nucleic acids; and determining a difference in the profile of the cells subjected to two different pharmaceutical conditions of the plurality of conditions. In some embodiments, the method of pharmaceutical screening comprises: providing a plurality of partitions each with a corresponding pharmaceutical condition of a plurality of pharmaceutical conditions, wherein partitions of the plurality of partitions each comprises one or more cells, or lysates thereof, subjected to the corresponding pharmaceutical condition of the partition; barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence; subjecting the plurality of barcoded nucleic acids, or products thereof, to sequencing to obtain sequences of the barcoded nucleic acids; determining a profile of the one or more cells from the sequences of the barcoded nucleic acids; and determining a difference in the profile of the cells subjected to two different pharmaceutical conditions of the plurality of conditions. The plurality of pharmaceutical conditions can comprise one or more pharmaceutical agents each at one or more concentrations, one or more control conditions, or combinations thereof. The one or more control conditions can comprise a solvent control, a negative control, a positive control, or a combination thereof.

Disclosed herein include methods of screening pharmaceutical agents. In some embodiments, a method of screening pharmaceutical agents comprises: introducing one or more cells into each partition of a plurality of partitions; subjecting the one or more cells in the partition to a pharmaceutical agent of a plurality of pharmaceutical agents; introducing a plurality of barcode molecules into the partition, wherein the barcode molecules each comprises a partition barcode sequence and a molecular barcode sequence; barcoding a plurality of target nucleic acids associated with the one or more cells in the partition using the plurality of the barcode molecules to generate a plurality of barcoded nucleic acids; subjecting the plurality of barcoded nucleic acids, or products thereof, to sequencing to obtain sequences of the barcoded nucleic acids; determining a profile of the one or more cells from the sequences of the barcoded nucleic acids; and analyzing the profile to determine an effect of the pharmaceutical agent on the one or more cells. In some embodiments, the method of screening pharmaceutical agents, comprises: introducing one or more cells into each partition of a plurality of partitions; subjecting the one or more cells in the partition to a pharmaceutical agent of a plurality of pharmaceutical agents; introducing a plurality of barcode molecules into the partition, wherein the barcode molecules each comprises a partition barcode sequence and a molecular barcode sequence; barcoding a plurality of target nucleic acids associated with the one or more cells in the partition using the plurality of the barcode molecules to generate a plurality of barcoded nucleic acids; subjecting the plurality of barcoded nucleic acids, or products thereof, to sequencing to obtain sequences of the barcoded nucleic acids; and analyzing the sequences of the barcoded nucleic acids to determine an effect of the pharmaceutical agent on the one or more cells. In some embodiments, the method of screening pharmaceutical agents comprises: introducing one or more cells into each partition of a plurality of partitions; subjecting the one or more cells in the partition to a pharmaceutical agent of a plurality of pharmaceutical agents; introducing a plurality of barcode molecules into the partition, wherein the barcode molecules each comprises a partition barcode sequence and a molecular barcode sequence; barcoding a plurality of target nucleic acids associated with the one or more cells in the partition using the plurality of the barcode molecules to generate a plurality of barcoded nucleic acids; and analyzing the plurality of barcoded nucleic acids to determine an effect of the pharmaceutical agent on the one or more cells. In some embodiments, the method of screening pharmaceutical agents comprises: introducing one or more cells into each partition of a plurality of partitions; subjecting the one or more cells in the partition to a pharmaceutical agent of a plurality of pharmaceutical agents; barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence; and analyzing the plurality of barcoded nucleic acids to determine an effect of the pharmaceutical agent on the one or more cells. In some embodiments, the method of screening pharmaceutical agents comprises: providing a plurality of partitions each comprising one or more cells subjected to a pharmaceutical agent of a plurality of pharmaceutical agents; barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence; and analyzing the plurality of barcoded nucleic acids to determine an effect of the pharmaceutical agent on the one or more cells.

In some embodiments, analyzing the plurality of barcoded nucleic acids comprises analyzing the sequences of the barcoded nucleic acids to determine an effect of the pharmaceutical agent on the one or more cells. In some embodiments, analyzing the sequences of the barcoded nucleic acids comprises: determining a profile of the one or more cells from the sequences of the barcoded nucleic acids; and analyzing the profile to determine an effect of the pharmaceutical agent on the one or more cells. In some embodiments, analyzing the profile comprises determining a difference between the profile and another profile, the difference being the effect of the pharmaceutical agent on the one or more cells, optionally the method comprises receiving the other profile or the other profile is the profile of the one or more cells in another partition of the plurality of partitions whose associated plurality of target nucleic acids is barcoded to generate a plurality of barcoded nucleic acids which is analyzed to determine the other profile, optionally the profile and the other profile are determined from similar numbers of cells.

In some embodiments, the method comprises releasing the plurality of target nucleic acids associated with the one or more cells in the partition prior to barcoding the plurality of target nucleic acids. Releasing the plurality of target nucleic acids associated with the one or more cells can comprise lysing the plurality of cells. In some embodiments, the plurality of target nucleic acids comprise deoxyribonucleic acid (DNA), genomic DNA (gDNA), ribonucleic acid (RNA), and/or messenger RNA (mRNA). The barcode molecule can further comprise a target binding sequence. The target binding sequence can, for example, comprise a poly(dT) sequence, a sequence capable of hybridizing to the plurality of target nucleic acids, or a combination thereof.

The method can, for example, further comprises introducing the plurality of the barcode molecules into the partition prior to subjecting the one or more cells in the partition with the pharmaceutical agent. In some embodiments, the method further comprises introducing the plurality of the barcode molecules into the partition after subjecting the one or more cells in the partition with the pharmaceutical agent. Barcoding the plurality of target nucleic acids can comprise a reverse transcription reaction, thereby generating a plurality of barcoded nucleic acids comprising complementary deoxyribonucleic acid (cDNA). In some embodiments, barcoding the plurality of target nucleic acids comprises extending the plurality of barcode molecules using the plurality of target nucleic acids as templates to generate the plurality of barcoded nucleic acids comprising a plurality of single-stranded barcoded nucleic acids, optionally hybridized to the plurality of target nucleic acids in the partition.

The method can further comprise introducing a plurality of template switching oligonucleotides into the partition, where barcoding the plurality of target nucleic acids comprises extending the plurality of barcode molecules using the plurality of target nucleic acids and the plurality of template switching oligonucleotides as templates to generate the plurality of barcoded nucleic acids comprising a plurality of single-stranded barcoded nucleic acids. In some embodiments, the method comprises introducing a plurality of extension primers to the partition, and wherein barcoding the plurality of target nucleic acids comprises extending the plurality of extension primers using the plurality of target nucleic acids as templates and the plurality of barcode molecules as template switching oligonucleotides to generate the plurality of barcoded nucleic acids comprising a plurality of single-stranded barcoded nucleic acids. In some embodiments, each of the plurality of single-stranded barcoded nucleic acids is hybridized to one of the plurality of target nucleic acids and one of the plurality of template switching oligonucleotides in the partition.

The method can, for example, further comprise removing the plurality of target nucleic acids and the plurality of template switching oligonucleotides hybridized to the single-stranded barcoded nucleic acids, optionally wherein removing the plurality of target nucleic acids comprises denaturation, thermal denaturation, digesting, or hydrolyzing the plurality of target nucleic acids. In some embodiments, each of the plurality of single-stranded barcoded nucleic acid comprises a sequence of a barcode molecule of the plurality of barcode molecules, a sequence of a target nucleic acid of the plurality of target nucleic acids, a sequence of a template switching oligonucleotide of the plurality of template switching oligonucleotides, and/or a sequence of an extension primer of the plurality of extension primers. A sequence can be the original sequence or a complementary sequence of the original sequence, such as the sequence of the reverse complement of the original sequence.

The method can, for example, further comprise amplifying the plurality of barcoded nucleic acids to generate a plurality of double-stranded barcoded nucleic acids in the partition using the single-stranded barcoded nucleic acids as templates. In some embodiments, amplifying the plurality of barcoded nucleic acids comprises amplifying the plurality of barcoded nucleic acids in the partition to generate the plurality of double-stranded barcoded nucleic acids, wherein the plurality of target nucleic acids in a partition are barcoded and the plurality of barcoded nucleic acids generated are then amplified in the same partition, and/or wherein the plurality of target nucleic acids in a partition are barcoded and the plurality of barcoded nucleic acids generated are then amplified in the same reaction. In some embodiments, each of the plurality of barcode molecules comprises a primer sequence, optionally wherein the primer sequence comprises a PCR primer sequence, wherein amplifying the plurality of barcoded nucleic acids comprises amplifying the plurality of barcoded nucleic acids using the primer sequences in single-stranded barcoded nucleic acids of the plurality of single-stranded barcoded nucleic acids, or products thereof.

The method can, for example, further comprise pooling the plurality of barcoded nucleic acids, or products thereof, in each of the plurality of partitions to generate pooled barcoded nucleic acids, wherein subjecting the plurality of barcoded nucleic acids, or products thereof, to sequencing comprises subjecting the pooled barcoded nucleic acids, or products thereof, to sequencing, optionally wherein pooling the plurality of barcoded nucleic acids, or products thereof, comprises pooling the plurality of double-stranded barcoded nucleic acids in each of the plurality of partitions to generate the pooled barcoded nucleic acids.

The method can, for example, further comprise fragmenting the pooled barcoded nucleic acids to generate fragmented barcoded nucleic acids to generate fragmented barcoded nucleic acids prior to subjecting the plurality of barcoded nucleic acids, or products thereof, to sequencing. In some embodiments, fragmenting the pooled barcoded nucleic acids comprises enzymatic fragmentation, physical fragmentation, or a combination thereof. In some embodiments, the enzymatic fragmentation comprises the use of one or more restriction enzymes.

The method can, for example, further comprise performing an amplification reaction (e.g., polymerase chain reaction) in bulk on the pooled barcoded nucleic acids, or the fragmented barcoded nucleic acids, to generate amplified barcoded nucleic acids. In some embodiments, performing the amplification reaction in bulk is subsequent to fragmenting the pooled barcoded nucleic acids. In some embodiments, the amplified barcoded nucleic acids comprise a sequence for attaching the amplified barcoded nucleic acids to a flow well. In some embodiments, the sequence for attaching the amplified barcoded nucleic acids to the flow well is a P5 sequence, a P7 sequence, or a portion thereof. In some embodiments, the amplified barcoded nucleic acids comprise a sequencing primer sequence.

The profile can comprise a multi-omics profile (e.g., a genomics profile, a proteomics profile, a transcriptomics profile, an epigenomics profile, a metabolomics profile, a chromatics profile, a protein expression profile, a cytokine secretion profile, or a combination thereof). In some embodiments, the profile comprises an expression of a target nucleic acid of the plurality of target nucleic acids, optionally wherein the expression of the target nucleic acid comprises an abundance of the target nucleic acid, optionally wherein the abundance of the target nucleic acid comprises an abundance of molecules of the target nucleic acid barcoded using the barcode molecules, optionally wherein the abundance of the molecules of the target nucleic acid comprises a number of occurrences of the molecules of the target nucleic acid, optionally wherein the number of occurrences of the molecules of the target nucleic acid is, is indicated by, or is determined using, a number of the barcoded nucleic acids comprising a sequence of the target nucleic acid and different molecular barcode sequences in the sequences of the barcoded nucleic acids.

In some embodiments, at least two of the partition barcode sequences of the plurality of barcode molecules in the same partition are identical. In some embodiments, the partition barcode sequences of at least one barcode molecules in at least two different partitions are different. In some embodiments, at least two of the molecular barcode sequences of the plurality of barcode molecules in a partition comprise different molecular barcode sequences, optionally wherein the molecular barcode sequences are unique molecular identifier. In some embodiments, each of the plurality of barcode molecules comprises a primer sequence (e.g., a sequencing primer sequence). Exemplary sequencing primer sequence include a Read 1 sequence, a Read 2 sequence, or a portion thereof.

The one or more cells can comprise at least 10, 100, 1000, or 10000 cells. In some embodiments, the one or more cells are obtained from, cultured from, derived from, or progenies of cells cultured from, a cell sample. The cell sample can be, for example, a clinical sample or a derivative thereof; a biological sample or a derivative thereof; a forensic sample or a derivative thereof; an environmental sample or a derivative thereof, or a combination thereof. The cell sample can be collected from blood, urine, serum, lymph, saliva, anal, and vaginal secretions, perspiration, and/or semen of any organism. In some embodiments, the cell sample is obtained from skin, bone, hair, brain, liver, heart, kidney, spleen, pancreas, stomach, intestine, bladder, lung, and/or esophagus of any organism. In some embodiments, the cells are cultured cells. In some embodiments, the cells comprise immune cells, fibroblast cells, stem cells, or cancer cells.

In some embodiments, introducing the plurality of barcode molecules to the partition comprises introducing a particle comprising the plurality of barcode molecules to the partition. The plurality of barcode molecules can be, for example, attached to, reversibly attached to, covalently attached to, or irreversibly attached to the particle.

In some embodiments, the particle is a gel particle, for example a hydrogel particle. In some embodiments, the gel particle is degradable upon application of a stimulus including but not limited to a thermal stimulus, a chemical stimulus, a biological stimulus, a photo-stimulus, or a combination thereof. In some embodiments, the particle is a solid particle and/or a magnetic particle. In some embodiments, the particle is retained in the partition by an external magnetic field during one or more steps of the method. In some embodiments, the particle comprises a paramagnetic material. The particle can, for example, have a size (e.g., diameter or a perimeter) of about 10 μm to about 100 μm.

The plurality of partitions can comprise a plurality of microwells of a microwell array. In some embodiments, the plurality of partitions comprises at least 100 partitions.

In some embodiments, the pharmaceutical agent comprises one or more therapeutic compounds, one or more hormones, one or more antibodies, one or more therapeutic peptides, one or more therapeutic nucleic acids, or combinations thereof. The pharmaceutical agent can be, or comprise, an anti-cancer compound. In some embodiments, the cells in two partitions are subject to two different pharmaceutical agents. In some embodiments, the cells in two partitions are subject to one pharmaceutical agent under different conditions. The conditions can, for example, comprise concentration of the pharmaceutical agent, dosage regimen of the pharmaceutical agent, temperature, duration, presence of one or more additional agents, or a combination thereof.

In some embodiments, the cells in the plurality of partitions are subject to at least 10 different pharmaceutical agents, one pharmaceutical agent under at least 10 different conditions (e.g., different concentration of the pharmaceutical agent), or both.

Disclosed herein also include kits for screening pharmaceutical agents. In some embodiments, the kit comprises: a plurality of barcode molecules; a microwell array comprising at least 100 microwells; and instructions to use the kit for pharmaceutical screening (or screening pharmaceutical agents) as described herein. In some embodiments, the kit further comprises one or more reagents used in the method.

The present disclosure provides an innovative, streamlined, and cost-effective RNA-seq method for drug screening that combines sample barcoding and one step Reverse Transcription-Polymerase Chain Reaction (RT-PCR) to simultaneously constructs RNA-seq libraries from tens to hundreds of samples. Barcode molecules (such as barcoding oligos) are designed to distinguish different samples in different partitions, such as different samples on a 96- or 384-well PCR plate, with each well containing one sample either untreated or treated with different types or concentrations of drugs. The sample (or cell) barcodes are added, in some embodiments, as part of the cDNA during a combined cell lysis, RT, and PCR process that is performed in the same reaction system and in the same well. This method allowed for pooling different samples of multiple-drug treatments in one subsequent library construction step, which can be used to reveal a unique transcriptome response for each drug and target-specific gene expression signatures, greatly accelerating the process of drug screening and reducing the costs. Reduced steps in the whole process also makes it easier to be automated. The approach of using tens to hundreds pooled, barcoded samples for library construction reduces the library construction costs by tens to hundreds of folds, reduces the required amounts of start materials and reagents, and improves sequencing capacity, making it cost effective.

Target-based drug discovery relies heavily on singular readouts such as reporter gene expression or modification of enzymatic activity in response to small molecule treatment. The present disclosure provides a more comprehensive and less-biased screening methods that combine aspects of both target-based and phenotypic screening.

In some embodiments, the method for high-throughput transcriptome profiling of drug screening in drug discovery comprises:
(a) distinguishing different samples and unique transcripts by barcoding oligo(dT) primer;
(b) reverse transcribing and enriching whole transcriptome in one step to generate cDNA;
(c) pooling up to 96 samples in one experiment;
(d) amplifying the cDNA; and/or
(e) analyzing the amplified cDNA.

In some embodiments, the method comprises performing cell lysis using chemical reagents.

In some embodiments, the barcoding oligo(dT) primer additionally comprises a sequence that can be used as PCR primer-binding sequence for amplification of the cDNA.

In some embodiments, the barcoding oligo(dT) primer comprises a unique molecular index (UMI) sequence that can be used to quantify the cDNA.

In some embodiments, the reverse transcription and the cDNA amplification are performed in a one-step RT-PCR.

In some embodiments, the analysis of the amplified cDNA comprises sequencing.

In some embodiments, the present disclosure provides a product that includes reagents needed to perform the high-throughput drug screening method disclosed herein.

### BRIEF DESCRIPTION OF THE DRAWINGS

FIG. 1A shows a representative workflow of a drug screening process disclosed herein. FIG. 1B shows a representative drug screening process using barcode molecules. FIGS. 1C and 1D show a schematic diagram of an experimental process that includes treatment of cells with different drugs and performing nucleic acid analysis using barcode molecules.

FIG. 2 shows quantification of gene expression levels in A549 cells after treatment with different drugs.

FIG. 3 shows analysis of differentially expressed genes in cells treated with different drugs.

FIG. 4 shows analysis of regulation of biological processes in cells treated with different drugs.

FIG. 5 shows results of a parallel correlation analysis between samples.

FIG. 6 shows results of a differential gene analysis.

FIG. 7 shows results of an enrichment (GO path) analysis of regulation of epithelial cell migration.

FIG. 8 shows results of a parallel correlation analysis.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Provided include methods, reagents, compositions, systems and kits for performing high-throughput pharmaceutical screening.

### Method of Screening

Disclosed herein include methods of pharmaceutical screening. The method can, for example, comprise introducing one or more cells into each partition of a plurality of partitions, subjecting the one or more cells in the partition to a pharmaceutical condition of a plurality of pharmaceutical conditions, and introducing a plurality of barcode molecules into the partition. The barcode molecules can, for example, each comprise a partition barcode sequence and a molecular barcode sequence. The method can further comprise barcoding a plurality of target nucleic acids associated with the one or more cells in the partition using the plurality of the barcode molecules to generate a plurality of barcoded nucleic acids, and optionally subjecting the plurality of barcoded nucleic acids, or products thereof, to sequencing to obtain sequences of the barcoded nucleic acids. The method can, for example, comprise determining a profile of the one or more cells from the sequences of the barcoded nucleic acids, and optionally determining a difference in the profile of the cells subjected to two different pharmaceutical conditions of the plurality of conditions.

The method of pharmaceutical screening can, in some embodiments, comprise providing a plurality of partitions each comprising one or more cells, or lysates thereof, subjected to a pharmaceutical condition of a plurality of pharmaceutical conditions; and barcoding a plurality of target nucleic acids associated with the one or more cells in the partition using a plurality of the barcode molecules to generate a plurality of barcoded nucleic acids. The one or more cells, in some embodiments, have been subjected to the pharmaceutical condition of a plurality of pharmaceutical conditions prior to being introduced into the plurality of partitions. In some embodiments, the one or more cells are subjected to the pharmaceutical condition of a plurality of pharmaceutical conditions after the cells are introduced into the plurality of partitions. The barcode molecules can, for example, each comprise a partition barcode sequence and a molecular barcode sequence. The method can further comprise subjecting the plurality of barcoded nucleic acids, or products thereof, to sequencing to obtain sequences of the barcoded nucleic acids; and optionally determining a profile of the one or more cells from the sequences of the barcoded nucleic acids. The method, in some embodiments, comprises determining a difference in the profile of the cells subjected to two different pharmaceutical conditions of the plurality of conditions.

In some embodiments, the method of pharmaceutical screening comprises providing a plurality of partitions each with a corresponding pharmaceutical condition of a plurality of pharmaceutical conditions. The partitions of the plurality of partitions can each comprise one or more cells, or lysates thereof, subjected to the corresponding pharmaceutical condition of the partition. The one or more cells, in some embodiments, have been subjected to the pharmaceutical condition of a plurality of pharmaceutical conditions prior to being introduced into the plurality of partitions. In some embodiments, the one or more cells are subjected to the pharmaceutical condition of a plurality of pharmaceutical conditions after the cells are introduced into the plurality of partitions. The method can further comprise barcoding a plurality of target nucleic acids associated with the one or more cells in the partition using a plurality of the barcode molecules to generate a plurality of barcoded nucleic acids. The barcode molecules can, for example, each comprise a partition barcode sequence and a molecular barcode sequence. The method can further comprise subjecting the plurality of barcoded nucleic acids, or products thereof, to sequencing to obtain sequences of the barcoded nucleic acids. The method can further comprise determining a profile of the one or more cells from the sequences of the barcoded nucleic acids, and optionally determining a difference in the profile of the cells subjected to two different pharmaceutical conditions of the plurality of conditions.

As described herein, a pharmaceutical condition can comprise a condition in which a pharmaceutical agent is present (e.g., at various concentrations or with different solvents) or absent. In some embodiments, the plurality of pharmaceutical conditions comprise one or more pharmaceutical agents each at one or more concentrations. In some embodiments, the plurality of pharmaceutical conditions comprise one or more conditions that can affect an outcome (including but not limited to a phenotypic response (e.g., changes in cell morphology), a genotypic response (e.g., gene mutation or epigenetic changes), response at transcriptional level, response at translational level, or any combination thereof) of the cells under the pharmaceutical condition. The conditions can include, but are not limited to, concentration of a pharmaceutical agent, dosage regimen of a pharmaceutical agent, presence and concentration of an additional pharmaceutical agent, temperature, duration, solvent control, negative control, and positive control. The one or more control conditions can comprise a solvent control, a negative control, and/or a positive control.

In some embodiments, subjecting the one or more cells (e.g., cells in a partition) to a pharmaceutical condition comprises subjecting the cells to one or more pharmaceutical agents. Subjecting the cells to a pharmaceutical agent can also be referred to herein as treating the cells with the pharmaceutical agent or a treatment of the cells by the pharmaceutical agent. The cells after being subject to a pharmaceutical agent can also be referred to herein as cells treated with the pharmaceutical agent or treated cells. Subjecting the cells to a pharmaceutical agent can comprise, for example, contacting the pharmaceutical agent with the cell in an aqueous medium. In some embodiments, the contacting comprises mixing the pharmaceutical agent with the cells in an aqueous medium. The aqueous medium can comprise water, a buffer, a culture medium in which the cells are cultured, or a combination thereof. The pharmaceutical agent can be dissolved in a solvent prior to contacting or mixing with the cells. The solvent can be, or comprise, water, an organic solvent such as an alcohol (e.g., ethanol, isopropanol, tetrahydrofurfuryl alcohol), a glycol (e.g., propylene glycol, polyethylene glycols, glycerin, diglyme), a ketone (e.g., acetone), dimethyl sulfoxide (DMSO), an oil (e.g., canola oil, corn oil, mineral oil), or a combination thereof. In some embodiments, the solvent comprises water, DMSO, or a combination thereof. In some embodiments, the pharmaceutical agent is dissolved in a solvent to form a solution, and the solution is added to the one or more cells in an aqueous medium in a partition.

The concentration of the pharmaceutical agent to which the cells are subjected to can vary, for example be, be about, be at least, be at least about, be at most, or be at most about, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, or a number or a range between any two of these values. For example, the concentration of the pharmaceutical agent can be 50 nM to 100 μM.

The pharmaceutical condition can comprise a temperature in which a pharmaceutical agent is present (e.g., at various temperature at the same or different concentration for the pharmaceutical agent) or absent. The temperature, as a condition, refers to the temperature under which the cells are subject to the pharmaceutical condition or the pharmaceutical agent. The temperature can vary, for example be, be about, be at least, be at least about, be at most, or be at most about, 0° C., 5° C., 10° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 45° C., 50° C., 55° C., 60° C., 65° C., 70° C., or a number or a range between any two of these values. For example, the temperature can be 15° C. to 40° C.

The pharmaceutical condition can comprise a duration (i.e., time period) during which the cells are subject to, for example, a pharmaceutical agent. The duration can vary, for example, be, be about, be at least, be at least about, be at most, or be at most about, 1 second, 5 seconds, 10 seconds, 15 seconds, 30 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 20 hours, 30 hours, 40 hours, 50 hours, 60 hours, 70 hours, 80 hours, 90 hours, 100 hours, 120 hours, 150 hours, 200 hours, 250 hours, 300 hours, or a number or a range between any two of these values. For example, the duration can be 30 minutes to 50 hours.

Dosage regimen of a pharmaceutical agent can comprise, for example, multiple doses of the pharmaceutical agent provided to the cells at various dosing intervals. For example, each dose of the pharmaceutical agent can be provided to the cells after each interval according to a schedule of predetermined intervals throughout the duration as disclosed herein. The concentration of the pharmaceutical agent provided to the cells after each interval can be identical or different. The intervals can vary, for example, be, be about, be at least, be at least about, be at most, or be at most about, 10 seconds, 30 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, or a number or a range between any two of these values. For example, the interval can be 1 minute to 5 hours. The number of intervals in the duration can be, be about, be at least, be at least about, be at most, or be at most about, 2, 3, 4, 5, 6, 7, 8, 9, 10, 20, 30, 40, 50, 60, 70, 80, 90, 100, or a number or a range between any two of these values.

In some embodiments, subjecting the one or more cells (e.g., cells in a partition) to a pharmaceutical condition comprises subjecting the cells to one or more conditions that can provide a comparison and/or validation of the methods as disclosed herein. The conditions can comprise a solvent control (e.g., DMSO), for example, to provide a control for the solvent used to dissolve a pharmaceutical agent. The condition can be, for example a control condition. The control condition can comprise a negative control (e.g. a cell free sample), for example, to provide a background level of an outcome of the cell. The control conditions can comprise a positive control (e.g., cells without treatment by a pharmaceutical agent), for example, to validate the effectiveness of a method.

In some embodiments, the cells produce a response as a result of being subject to (e.g., being contacted with) a pharmaceutical agent. The response of the cells can indicate an effect of the pharmaceutical agent on the cells. The pharmaceutical agent can cause, for example, binding of the pharmaceutical agent to a receptor on a surface of a cell, internalization of the pharmaceutical agent into a cell, activation of a signaling cascade, inhibition of an enzyme, activation of an enzyme, or a combination thereof. The response of the cell can comprise a modification of a gene expression profile of the cell. The response can comprise a change of expression levels of one or more genes of the cell. The response can comprise an upregulation or a downregulation of expression of one or more genes of the cell.

In some embodiments, the method of screening pharmaceutical agents comprises introducing one or more cells into each partition of a plurality of partitions; and subjecting the one or more cells in the partition to a pharmaceutical agent of a plurality of pharmaceutical agents. The method can further comprise introducing a plurality of barcode molecules into the partition. The barcode molecules can, for example, each comprise a partition barcode sequence and a molecular barcode sequence. The method can further comprise barcoding a plurality of target nucleic acids associated with the one or more cells in the partition using the plurality of the barcode molecules to generate a plurality of barcoded nucleic acids; and optionally subjecting the plurality of barcoded nucleic acids, or products thereof, to sequencing to obtain sequences of the barcoded nucleic acids. The method can further comprise determining a profile of the one or more cells from the sequences of the barcoded nucleic acids; and optionally analyzing the profile to determine an effect of the pharmaceutical agent on the one or more cells.

In some embodiments, the method of screening pharmaceutical agents comprises introducing one or more cells into each partition of a plurality of partitions; and subjecting the one or more cells in the partition to a pharmaceutical agent of a plurality of pharmaceutical agents. The method can comprise introducing a plurality of barcode molecules into the partition. The barcode molecules can each comprise a partition barcode sequence and a molecular barcode sequence. The method can comprise barcoding a plurality of target nucleic acids associated with the one or more cells in the partition using the plurality of the barcode molecules to generate a plurality of barcoded nucleic acids. The method can comprise subjecting the plurality of barcoded nucleic acids, or products thereof, to sequencing to obtain sequences of the barcoded nucleic acids. In some embodiments, the method comprises analyzing the sequences of the barcoded nucleic acids to determine an effect of the pharmaceutical agent on the one or more cells. In some embodiments, the method comprises analyzing the plurality of barcoded nucleic acids to determine an effect of the pharmaceutical agent on the one or more cells.

In some embodiments, the method of screening pharmaceutical agents comprises providing a plurality of partitions each comprising one or more cells subjected to a pharmaceutical agent of a plurality of pharmaceutical agents; and barcoding a plurality of target nucleic acids associated with the one or more cells in the partition using a plurality of the barcode molecules to generate a plurality of barcoded nucleic acids. The barcode molecules can each comprises a partition barcode sequence and a molecular barcode sequence. In some embodiments, the method comprises analyzing the sequences of the barcoded nucleic acids to determine an effect of the pharmaceutical agent on the one or more cells. In some embodiments, the method comprises analyzing the plurality of barcoded nucleic acids to determine an effect of the pharmaceutical agent on the one or more cells.

Analyzing the plurality of barcoded nucleic acids can, for example, comprise analyzing the sequences of the barcoded nucleic acids to determine an effect of the pharmaceutical agent on the one or more cells. In some embodiments, analyzing the sequences of the barcoded nucleic acids comprises determining a profile (e.g., an expression profile) of the one or more cells from the sequences of the barcoded nucleic acids, and optionally analyzing the profile to determine an effect of the pharmaceutical agent on the one or more cells.

In some embodiments, analyzing the profile comprises determining a difference between the profile and another profile, the difference being the effect of the pharmaceutical agent on the one or more cells. The other profile can be an existing profile. The other profile can be determined as described herein. The method can, for example, comprise receiving the other profile or the other profile is the profile of the one or more cells in another partition of the plurality of partitions whose associated plurality of target nucleic acids is barcoded to generate a plurality of barcoded nucleic acids which is analyzed to determine the other profile. For example, the profile can be a profile of the cells after being subject to a pharmaceutical agent, and the other profile can be a profile of the cells before being subject to the pharmaceutical agent, after being subject to the pharmaceutical agent at a different concentration, or after being subject to a different pharmaceutical agent at a same concentration. Further, the profile and the other profile can be determined from similar number of cells. For example, the percentage difference between the numbers of cells from which the profile and the other profile are determined can be, be about, be at least, be at least about, be at most, or be at most about, 0.1%, 0.5%, 1%, 2%, 3%, 4%, 5%, 6%, 7%, 8%, 9%, 10%, 15%, 20%, 25%, 30%, 35%, 40%, 45%, 50%, or a number or a range between any two of these values. In some embodiments, the difference between the numbers of cells from which the profile and the other profile are determined can be, be about, be at least, be at least about, be at most, or be at most about, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, or a number or a range between any two of these values. In some embodiments, the percentage difference between the numbers of cells from which the profile and the other profile are determined is 0.1% to 5%. In some embodiments, the difference between the numbers of cells from which the profile and the other profile are determined is 1 to 5000.

The cells in different partitions can be subject to identical or different pharmaceutical conditions (e.g., different pharmaceutical agents). In some embodiments, the cells in two partitions are subject to two different pharmaceutical agents. The number of different pharmaceutical agents can be different in different embodiments. For example, the number of different pharmaceutical agents can be, be about, be at least, be at least about, be at most, or be at most about, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 400, 500, 600, 700, 800, 900, 1000, 2000, 3000, 4000, 5000, 6000, 7000, 8000, 9000, 10000, or a number or a range between any two of these values. In some embodiments, the cells in the plurality of partitions are subject to at least 10 different pharmaceutical agents.

The cells in different partitions can be subject to identical or different conditions as disclosed herein. In some embodiments, the cells in two partitions are subject to one pharmaceutical agent under different conditions. The different conditions can comprise, for example, concentration of the pharmaceutical agent, dosage regimen of the pharmaceutical agent, temperature, duration, presence of one or more additional pharmaceutical agents, or a combination thereof. The number of different conditions can vary, for example, the number of different conditions can be, be about, be at least, be at least about, be at most, or be at most about, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 400, 500, 600, 700, 800, 900, 1000, or a number or a range between any two of these values. In some embodiments, the cells in the plurality of partitions are subject to at least 10 different conditions.

In some embodiments, to simultaneously implement RNA-Seq under multiple drug treatment conditions, barcode molecules (or barcoding tags) are used to capture and distinguish mRNAs from samples treated with different drugs.

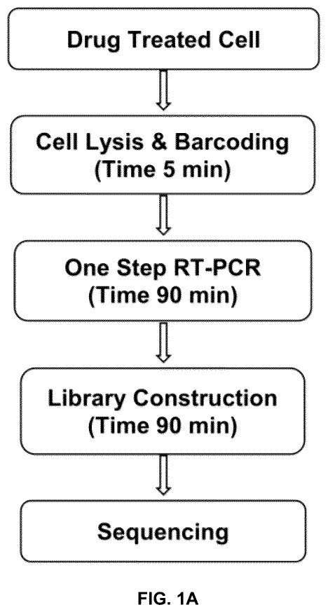

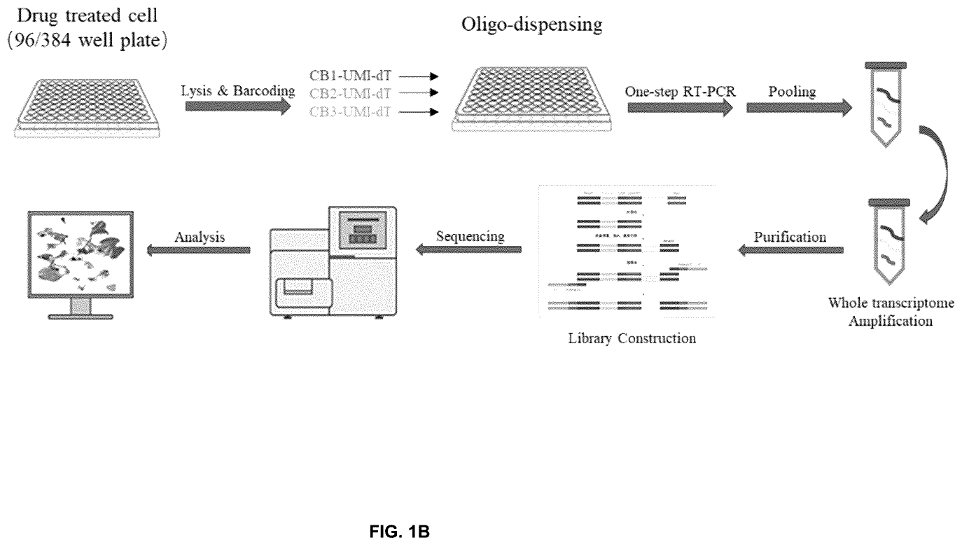

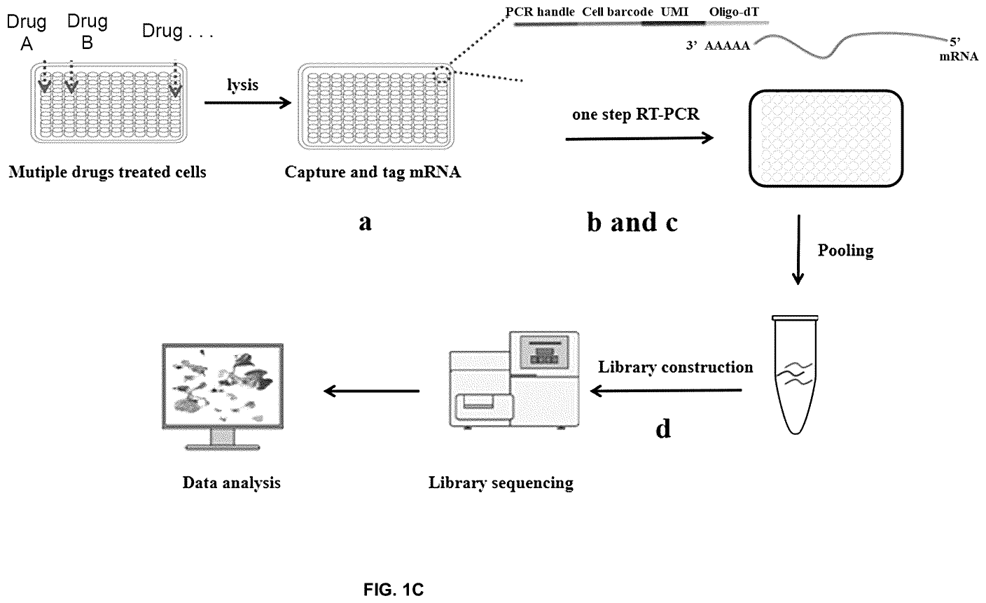

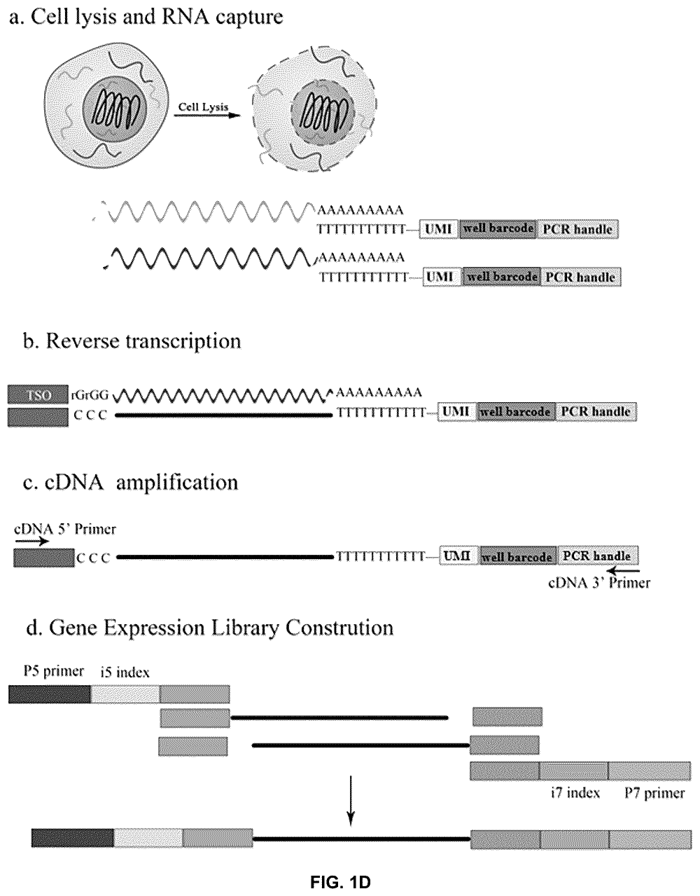

As described herein, barcode molecules comprising poly-dT sequences (also referred to herein as barcoding oligo-dT) can be used to tag multiple samples through mRNA captured. In some embodiments, the barcode molecules can comprise a PCR handle (or PCR primer binding) sequence, a partition barcode (e.g., a well position specific barcode sequence), a random DNA sequence (e.g., a molecular barcode sequence as unique molecular index (UMI)), and a target binding sequence (e.g., an oligo(dT) primer sequence.) The PCR handle sequence can act as priming site for RT reactions and PCR amplification reactions. The partition barcode can be used to label different samples in different wells (e.g., cells treated with different drugs). The UMI can be used to detect and quantify unique mRNA transcripts. After reverse transcribing the mRNA, RT enzyme adds oligo(dC) to the end of first-strand cDNA, which allows the template switching oligonucleotide (TSO) to bind. Samples can be pooled after the one step RT-PCR. After pre-amplification and tagmentation, paired end libraries can be sequenced (FIGS. 1A-1D).

The present disclosure provides an innovative, streamlined, and cost-effective RNA-seq method for drug screening that combines sample barcoding and one step Reverse Transcription-Polymerase Chain Reaction (RT-PCR) to simultaneously constructs RNA-seq libraries from tens to hundreds of samples.

By using barcoding oligo-dT to tag multiple samples through mRNA capture, the present method can greatly accelerate the process of drug screening at reduced costs. The present method can simplify RT and PCR amplification in a one-step reaction, thus reducing operation steps in the whole process and making it easier to be automated. The present approach allows for library construction for tens to hundreds pooled, barcoded samples, which reduces the library construction costs by tens to hundreds of folds, reduces the required amounts on start materials and reagents, and improves sequencing capacity, making it more cost effective. Further, the present method can add sample barcodes as part of the cDNA in a combined cell lysis, RT, and PCR process in the same reaction system and in the same well. The present method allows for pooling different samples of multiple-drug treatments in one subsequent library construction step, which can reveal a unique transcriptome response for each drug and target-specific gene expression signatures. In the present method, UMI can be added as part of the cDNA. Sequencing with UMIs can reduce the rate of false-positive variant calls and increase sensitivity of variant detection.

### Partition

A partition as used herein refers to a part, a portion, or a division sequestered from the rest of the parts, portions, or divisions. A partition can be formed through the use of wells, microwells, multi-well plates, microwell arrays, microfluidics, dilution, dispensing, droplets, or any other means of sequestering one fraction of a sample from another. In some embodiments, a partition is a well, a droplet or a microwell. In some embodiments, a partition is a well in a multi-well plate.

The plurality of partitions can comprise at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, or 500000 partitions. In some embodiments, the plurality of partitions comprises at least 100 partitions.

In some embodiments, one or more cells and a pharmaceutical agent are mixed in a partition, thereby subjecting the one or more cells to the pharmaceutical agent. In some embodiments, the one more cells are lysed in the same partition where the one or more cells are subject to the pharmaceutical agent. In some embodiments, the barcoding and RT-PCR reactions are performed in the same partition where the one or more cells are subject to the pharmaceutical agent. In some embodiments, the cells after being subject to the pharmaceutical agent are transferred to an unoccupied partition, in which the barcoding and/or RT-PCR reactions are performed. In some embodiments, the lysed cells are transferred to an unoccupied partition, in which the barcoding and/or RT-PCR reactions are performed.

The barcode molecules can be introduced to the partitions directly. The barcode molecules can be attached to a particle (e.g., a bead), and introducing the barcode molecules can comprise introducing the particle to a partition. In some embodiments, barcode molecules can be introduced into the partitions (e.g., microwells or wells) by attaching or synthesizing the plurality of barcode molecules onto the surface of the partitions.

Microwell Array

In some embodiments, the plurality of plurality of partitions comprise a plurality of microwells of a microwell array. The microwell array can comprise different numbers of microwells in different implementations. In some embodiments, the microwell array can comprise, comprise about, comprise at least, comprise at least about, comprise at most, or comprise at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values, microwells. The microwells can be arranged into rows and columns, for example. The number of microwells in a row (or a column) can be, be about, be at least, be at least about, be at most, or be at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, or a number or a range between any two of these values. Adjacent rows (or columns) of microwells can be aligned or staggered, for example.

The width, length, depth (or height), radius, or diameter of a microwell of the plurality of microwells can be different in different implementations. In some embodiments, the width, length, depth (or height), radius, or diameter of a microwell of the plurality of microwells can be, be about, be at least, be at least about, be at most, or be at most about, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm, or a number or a range between any two of these values. For example, the width of a microwell of the plurality of microwells is 10 μm to 200 μm. As another example, the length of a microwell of the plurality of microwells can be 10 μm to 200 μm. As a further example, the depth of a microwell of the plurality of microwells can be 5 μm to 500 μm. In the non-limiting exemplary embodiment, the width of a microwell is 10 μm, the length of a microwell is 20 μm to 100 μm, such as 20 μm, and the depth of a microwell is 5 μm to 10 μm. The shape of a microwell can be different in different embodiments. In some embodiments, a microwell has a circular, elliptical, square, rectangular, triangular, or hexagonal shape.

The volume of one, one or more, or each, of the plurality of microwells can be different in different embodiments. The volume of one, one or more, or each, of the plurality of microwells can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 10000000 nm³, 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm 3, or a number or a range between any two of these values. The volume of one, one or more, or each, of the plurality of microwells can be, be about, be at least, be at least about, be at most, or be at most about, 1 nanolitre (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, one or more, or each, of the plurality of microwells is about 1 nm3 to about 1000000 μm³.

The microwell array comprising a plurality of microwells can be formed from any suitable material as will be understood by a person of skill in the art. In some embodiments, a microwell array comprising a plurality of microwells can be formed from a material selected from the group consisting of silicon, glass, ceramic, elastomers such as polydimethylsiloxane (PDMS) and thermoset polyester, thermoplastic polymers such as polystyrene, polycarbonate, poly(methyl methacrylate) (PMMA), poly-ethylene glycol diacrylate (PEGDA), Teflon, polyurethane (PU), composite materials such as cyclic-olefin copolymer, and combinations thereof.

The cells can be partitioned into wells of a multi-well plate. The plate can comprise different numbers of wells in different embodiments. In some embodiments, the plate can comprise, comprise about, comprise at least, comprise at least about, comprise at most, or comprise at most about, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000, or a number or a range between any two of these values, wells. The wells can be arranged into rows and columns, for example. The number of wells in a row (or a column) can be, be about, be at least, be at least about, be at most, or be at most about, 2, 4, 6, 8, 10, 20, 30, 40, 50, 60, 70, 80, 90, or 100, or a number or a range between any two of these values. Adjacent rows (or columns) of microwells can be aligned or staggered, for example. In some embodiments, the plate comprise at least, at least about, at most, or at most about 50, 100, 500, 1000, 1500, or 3000 wells. In some embodiments, the plate comprises 96 wells (8×12) or 394 wells (16×24).

The width, length, depth (or height), radius, or diameter of a well of the plurality of wells can be different in different embodiments. For example, the width, length, depth (or height), radius, or diameter of a microwell of the plurality of wells can be, be about, be at least, be at least about, be at most, or be at most about, 0.2 mm, 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5, 9 mm, 9.5 mm, 10 mm, 11 mm, 12 mm, 13 mm, 14 mm, 15 mm, 16 mm, 17 mm, 18 mm, 19 mm, 20 mm, or a number or a range between any two of these values. For example, the width of a well of the plurality of wells is 0.5 mm to 15 mm. In some embodiments, the length of a microwell of the plurality of microwells can be 0.5 mm to 15 mm. In some embodiments, the depth of a well of the plurality of wells can be 0.5 mm to 15 mm. The shape of a well can be different in different embodiments, for example, the well can have a circular, elliptical, square, rectangular, triangular, or hexagonal shape.

The volume of one, one or more, or each, of the plurality of wells can be different in different embodiments. The volume of one, one or more, or each, of the plurality of microwells can vary, for example, be, be about, be at least, be at least about, be at most, or be at most about, 0.1 μL, 0.5 μL, 1 μL, 2 μL, 3 μL, 4 μL, 5 μL, 6 μL, 7 μL, 9 μL, 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1000 μL, 2000 μL, 3000 μL, 4000 μL, 5000 μL, 6000 μL, 7000 μL, 8000 μL, 9000 μL, or 10000 μL. For example, the volume of one, one or more, or each, of the plurality of microwells is about 1 μL to about 500 μL.

Partitions (e.g., microwells and wells) described above can be introduced with samples, free reagents, and/or reagents encapsulated in microcapsules. The reagents can comprise restriction enzymes, ligase, polymerase, fluorophores, oligonucleotide barcodes, oligonucleotide probes, adapters, buffers, dNTPs, ddNTPs, and other reagents required for performing the methods described herein.

### Partitioning Cells

The number of cells in each partition can vary, for example, be, be about, be at least, be at least about, be at most, or be at most about 1, 2, 3, 4, 5, 6, 7, 8, 9, 10, 15, 20, 25, 30, 35, 40, 45, 50, 100, 200, 300, 400, 500, 600, 700, 800, 900, 1000, 2000, 3000, 4000, 5000, or a number or a range between any two of these values. For example, the number of cells in each partition can be from 1 to 5. In some embodiments, the one or more cells comprise at most 5 cells, 8 cells, 10 cells, 50 cells, 75 cells, or 100 cells. In some embodiments, the cells are placed in wells of a multi-well plate, and the number of cells in each well is from 2 to 100.

In some embodiments, the cells are partitioned into a plurality of microwells of a microwell array. As a result of partitioning, the percentage of the plurality of partitions comprising a desired number of cell(s) (e.g., a single cell, at least 10 cells, at least 100 cells, at least 1000 cells, or at least 10000 cells), and optionally a single particle (e.g., a bead) can vary. For example, the percentage of the plurality of partitions comprising the desired number of cell(s) and optionally a single particle can be, be about, be at least, be at least about, be at most, or be at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, at least 10% of the plurality of partitions comprise a desired number of cell(s) and optionally a single particle.

The percentage of the plurality of partitions comprising no cell can be different in different embodiments. For example, the percentage of the plurality of partitions comprising no cell can be, be about, be at least, be at least about, be at most, or be at most about, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. In some embodiments, at most 50% of partitions of the plurality of partitions can comprise no cell of the plurality of cells.

The percentage of the plurality of partitions comprising more than the desired number of cell(s) can be different in different embodiments. For example, the percentage of the plurality of partitions comprising more than the desired number of cell(s) can be, be about, be at least, be at least about, be at most, or be at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. In some embodiments, at most 10% of partitions of the plurality of partitions can comprise more than the desired number of cell(s) of the plurality of cells.

### Target Nucleic Acids

As described herein, cells can be associated with target nucleic acids. For example, a cell can comprise one or more target nucleic acids (e.g., mRNA) or can be labeled with one or more target nucleic acids (e.g., directly, or indirectly through a binding moiety, such as an antibody conjugated with the nucleic acid). The target nucleic acids associated with the cell can be from, on the surface of, or binding to the surface of the cell. A target nucleic acid can have a sequence (e.g., an mRNA sequence, excluding the poly(A) tail).

The target nucleic acids associated with the cell can comprise deoxyribonucleic acid (DNA), ribonucleic acid (RNA), and/or any combination or hybrid thereof. The target nucleic acids can be single-stranded or double-stranded, or contain portions of both double-stranded or single-stranded sequences. The target nucleic acids can contain any combination of nucleotides, including uracil, adenine, thymine, cytosine, guanine, inosine, xanthine, hypoxanthine, isocytosine, isoguanine and any nucleotide derivative thereof. As used herein, the term “nucleotide” can include naturally occurring nucleotides and nucleotide analogs, including both synthetic and naturally occurring species. The target nucleic acids can be genomic DNA (gDNA), mitochondrial DNA (mtDNA), messenger RNA (mRNA), ribosomal RNA (rRNA), transfer RNA (tRNA), nuclear RNA (nRNA), small interfering RNA (siRNA), small nuclear RNA (snRNA), small nucleolar RNA (snoRNA), small Cajal body-specific RNA (scaRNA), microRNA (miRNA), double stranded (dsRNA), ribozyme, riboswitch or viral RNA, or any nucleic acids that may be obtained from a sample.

The plurality of target nucleic acids can, for example, comprise DNA, genomic DNA (gDNA), ribonucleic acid (RNA), and/or messenger RNA (mRNA). In some embodiments, the plurality of target nucleic acids comprises mRNA, for example a poly-adenylated mRNA.

### Barcoding

In the methods disclosed herein, barcode molecules (e.g., barcode molecules associated with particles) can be introduced into the partitions for barcoding target nucleic acids. For example, the amount of the barcode molecules added directly to a partition can be, be about, be at least, be at least about, be at most, or be at most about, 0.1 ng, 0.2 ng, 0.3 ng, 0.4 ng, 0.5 ng, 0.6 ng, 0.7 ng, 0.8 ng, 0.9 ng, 1 ng, 2 ng, 3 ng, 4 ng, 5 ng, 6 ng, 7 ng, 8 ng, 9 ng, 10 ng, 20 ng, 30 ng, 40 ng, 50 ng, 60 ng, 70 ng, 80 ng, 90 ng, 100 ng, 200 ng, 300 ng, 400 ng, 500 ng, 600 ng, 700 ng, 800 ng, 900 ng, 1000 ng, 2000 ng, 3000 ng, 4000 ng, 5000 ng, 6000 ng, 7000 ng, 8000 ng, 9000 ng, 10000 ng, 2000 ng, 3000 ng, 4000 ng, 5000 ng, 6000 ng, 7000 ng, 8000 ng, 90000 ng or a number or a range between any two of these values. In some embodiments, the barcode molecules can be introduced into the partitions (e.g. microwells or wells) by attaching or synthesizing the plurality of barcode molecules onto the surface of the partitions.

The barcode molecules introduced into the partitions (e.g., microwells or droplets) can be associated with particles (e.g., beads). In some embodiments, introducing the plurality of barcode molecules to the partition comprises introducing a particle comprising the plurality of barcode molecules to the partition. The particles can provide a surface upon which molecules, such as oligonucleotides, can be synthesized or attached. In some embodiments, the plurality of barcode molecules are attached to, reversibly attached to, covalently attached to, or irreversibly attached to the particle.

The particle can comprise, comprise about, comprise at least, comprise at least about, comprise at most, or comprise at most about, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 7000000, 8000000, 9000000, 10000000, 20000000, 50000000, 100000000, 200000000, 300000000, 400000000, 500000000, 600000000, 700000000, 800000000, 900000000, 1000000000, or a number or a range between any two of these values, barcode molecules. The attachment of barcode molecules to the particle can be covalent or non-covalent via non-covalent bonds such as ionic bonds, hydrogen bonds, or van der Waals interactions. The attachment can be direct to the surface of a particle or indirect through other oligonucleotide sequences attached to the surface of a particle.

The particle (e.g., a bead) can be dissolvable, degradable, or disruptable. A particle can be a gel particle such as a hydrogel particle. In some embodiments, the gel particle is degradable upon application of a stimulus. The stimulus can comprise a thermal stimulus, a chemical stimulus, a biological stimulus, a photo-stimulus, or a combination thereof. The particle can be a solid particle and/or a magnetic particle. In some embodiments, the particle is a magnetic particle. The magnetic particle can comprise a paramagnetic material coated or embedded in the magnetic particle (e.g. on a surface, in an intermediate layer, and/or mixed with other materials of the magnetic particle). A paramagnetic material refers to a material having a magnetic susceptibility slightly greater than 1 (e.g. between about 1 and about 5). A magnetic susceptibility is a measure of how much a material can become magnetized in an applied magnetic field. Paramagnetic materials include, but not limited to, magnesium, molybdenum, lithium, aluminum, nickel, tantalum, titanium, iron oxide, gold, copper, or a combination thereof. In some embodiments, the magnetic particle comprising barcode molecules can be immobilized or retained in a partition (such as a microwell or a well) by an external magnetic field, thereby retaining the barcode molecules in a partition. The magnetic particle comprising barcode molecules can be mobilized or released when the external magnetic field is removed.

In some embodiments, a particle can be immobilized or retained in a partition (e.g., a microwell or a well) through an interaction between two members of a binding pair. For example, the partition (e.g., microwell or well) can be coated with a capture moiety (e.g., a member of a binding pair) capable of binding with a binding moiety (the other member of the binding pair) comprised in or conjugated to a particle, such that the binding of the two moieties results in the attachment of the particle to the partition (e.g., microwell or well), thereby immobilizing or retaining the particle in the partition. For example, the surface of a partition (e.g., microwell or well) can be coated with streptavidin. The biotinylated particle can be attached to the surface of the partition (e.g., microwell or well) via streptavidin-biotin interaction.

Particles can be of uniform size or heterogeneous size. In some embodiments, one or more of the particles have a diameter of about, at least, at least about, at most, or at most about, 1 μm, 5 μm, 10 μm, 20 μm, 30 μm, 40 μm, 45 μm, 50 μm, 60 μm, 65 μm, 70 μm, 75 μm, 80 μm, 90 μm, 100 μm, 250 μm, 500 μm, or 1 mm.

In some embodiments, a particle can be sized such that at most one particle, not two particles, can fit one partition. A size or dimension (e.g., length, width, depth, radius, or diameter) of a particle can be different in different embodiments. For example, a size or dimension of one, or each, particle can be, be about, be at least, be at least about, be at most, or be at most about, 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. In some embodiments, a size or dimension of one, or each, particle is about 1 nm to about 100 μm. In some embodiments, the particle can have a dimension about 10 μm to about 100 μm. In some embodiments, the particle can have a dimension about 30 μm.

The volume of one, or each, particle can vary. The volume of one, or each, particle can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 10000000 nm³, 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm³, or a number or a range between any two of these values. The volume of one, or each, particle can be, be about, be at least, be at least about, be at most, or be at most about, 1 nanolitre (nL), 2 nL, 3 nL, 4 nL, 5 nL, 6 nL, 7 nL, 8 nL, 9 nL, 10 nL, 11 nL, 12 nL, 13 nL, 14 nL, 15 nL, 16 nL, 17 nL, 18 nL, 19 nL, 20 nL, 21 nL, 22 nL, 23 nL, 24 nL, 25 nL, 26 nL, 27 nL, 28 nL, 29 nL, 30 nL, 31 nL, 32 nL, 33 nL, 34 nL, 35 nL, 36 nL, 37 nL, 38 nL, 39 nL, 40 nL, 41 nL, 42 nL, 43 nL, 44 nL, 45 nL, 46 nL, 47 nL, 48 nL, 49 nL, 50 nL, 51 nL, 52 nL, 53 nL, 54 nL, 55 nL, 56 nL, 57 nL, 58 nL, 59 nL, 60 nL, 61 nL, 62 nL, 63 nL, 64 nL, 65 nL, 66 nL, 67 nL, 68 nL, 69 nL, 70 nL, 71 nL, 72 nL, 73 nL, 74 nL, 75 nL, 76 nL, 77 nL, 78 nL, 79 nL, 80 nL, 81 nL, 82 nL, 83 nL, 84 nL, 85 nL, 86 nL, 87 nL, 88 nL, 89 nL, 90 nL, 91 nL, 92 nL, 93 nL, 94 nL, 95 nL, 96 nL, 97 nL, 98 nL, 99 nL, 100 nL, or a number or a range between any two of these values. In some embodiments, the volume of one, or each, particle is about 1 nm³ to about 1000000 μm³.

The number of particles introduced into a plurality of partitions can be different in different embodiments. In some embodiments, the number of particles introduced into a plurality of partitions is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, or a number or a range between any two of these values.

In some embodiments, particles are introduced to the partitions such that the percentage of partitions each occupied with one particle is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, at least 80% of the plurality of partitions is each occupied with one particle.

In some embodiments, particles are introduced to the partitions such that the percentage of partitions with no particle is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. In some embodiments, at most 20% of the plurality of partitions contain no particle.

### Pharmaceutical Agents

Methods, reagents, compositions, systems, and kits disclosed herein can be used to screen pharmaceutical agents (e.g., drug candidates). The pharmaceutical agent can be or comprise, for example, one or more therapeutic compounds, one or more hormones, one or more antibodies, one or more therapeutic peptides, one or more therapeutic nucleic acids, or a combination thereof. In some embodiments, the pharmaceutical agent comprises one or more therapeutic compounds, for example known drugs.

In some embodiments, the pharmaceutical agent comprises an anti-cancer agent, such as an anti-cancer drug (including but not limited to, a small molecule drug or a biologics). Examples of anti-cancer drugs include, but are not limited to, abiraterone, afatinib, aminolevulinic acid, aprepitant, axitinib, azacitidine, belinostat, bendamustine, bevacizumab, bexarotene, bleomycin, bortezomib, bosutinib, busulfan, cabazitaxel, cabozantinib, capecitabine, carboplatin, carfilzomib, carmustine, ceritinib, cetuximab, chlorambucil, cisplatin, clofarabine, crizotinib, cyclophosphamide, cytarabine, dabrafenib, dacarbazine, dactinomycin, dasatinib, daunorubicin, decitabine, denosumab, dexrazoxane, docetaxel, dolastatins (e.g. monomethyl auristatin E), doxorubicin, enzalutamide, epirubicin, eribulin mesylate, erlotinib, etoposide, everolimus, floxuridine, fludarabine phosphate, fluorouracil, ganetespib, gefitinib, geldanamycin, gemcitabine, gemtuzumab ozogamicin, hexamethylmelamine, hydroxyurea, ibritumomab tiuxetan, ibrutinib, idelalisib, ifosfamide, imatinib, ipilimumab, irinotecan, ixabepilone, lapatinib, leucovorin calcium, lomustine, maytansinoids, mechlorethamine, melphalan, mercaptopurine, mertansine, mesna, methotrexate, mitomycin C, mitotane, mitoxantrone, nelarabine, nelfinavir, nilotinib, obinutuzumab, ofatumumab, omacetaxine mepesuccinate, oxaliplatin, paclitaxel, panitumumab, pazopanib, pegaspargase, pembrolizumab, pemetrexed, pentostatin, pertuzumab, plicanycin, pomalidomide, ponatinib hydrochloride, pralatrexate, procarbazine, radium 223 dichloride, ramucirumab, regorafenib, retaspimycin, ruxolitinib, semustine, siltuximab, sorafenib, streptozocin, sunitinib malate, tanespimycin, temozolomide, temsirolimus, teniposide, thalidomide, thioguanine, thiotepa, topotecan, toremifene, trametinib, trastuzumab, vandetanib, vemurafenib, vinblastine, vincristine, vinorelbine, vismodegib, vorinostat, and ziv-aflibercept. In some embodiments, the pharmaceutical agent comprises an inhibitor of lysine-specific demethylase (LSD1), including but are not limited to, capsaicin, biochanin A, salvianolic acid B, rosmarinic acid, dihydrotanshinone I, cryptotanshinone, tanshinone I, and isoquinoline alkaloids (e.g., epiberberine, columbamine, jatrorrhizine, berberine, and palmatine), tranylcypromine, or a pharmaceutically acceptable salt, hydrate, solvate or prodrug thereof.

In some embodiments, the pharmaceutical agent is or comprises an agent or compound for treating cardiovascular disease, including but not limited to, an angiotensin-converting enzyme (ACE) inhibitor (e.g., alacepril, benazepril, captopril, cilazapril, enalapril, fosinopril, imidapril, indolapril, lisinopril, perindopril, quinapril, ramipril, trandolapril, zofenopril), an angiotensin II receptor blocker (e.g., candesartan, eprosartan, fimasartan, irbesartan, losartan, olmesartan, telmisartan, valsartan), a beta blocker (e.g., acebutolol, alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol, indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol, sotalol, sulfinalol, talinolol, tertatolol, timolol, toliprolol, xibinolol), a diuretic (e.g., a thiazide or benzothiadiazine derivative (such as althiazide, bendroflumethazide, benzthiazide, benzylhydrochiorchlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (such as chlormerodrin, meralluride, mercamnphamide, mercaptomerin sodium, mercumallylic acid, mercumatilin dodium, mercurous chloride, mersalyl), a pteridine (such as furtherene, triamterene), a purine (such as acefylline, 7-morpholinomethyltheophylline, pamobrom, protheobromine, theobromine), a steroid (including aldosterone antagonists such as canrenone, oleandrin, spironolactone), a sulfonamide derivative (such as aceG6PDolamide, ambuside, azosemide, bumetanide, buG6PDolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (such as aminometradine, amisometradine), a potassium sparing antagonist (such as amiloride, triamterene), or a miscellaneous diuretic (such as aminozine, arbutin, chlorazanil, ethacrynic acid, etozolin, hydracarbazine, isosorbide, mannitol, metochalcone, muzolimine, perhexyline, ticrynafen, urea)), or a combination thereof.

In some embodiments, the pharmaceutical agent is or comprises an agent or compound for treating diabetes, including but not limited to, insulin, an alpha-glucosidase inhibitor, metaformin, a dopamine agonist (e.g. bromocriptine), a dipeptidyl peptidase-4 (DPP-4) inhibitor (e.g., alogliptin, anagliptin, berberine, dutogliptin, evogliptin, gemigliptin, gosogliptin, linagliptin, omarigliptin, saxagliptin, sitagliptin, trelagliptin, vildagliptin), a glucagon-like peptide-1 (GLP-1) receptor agonist (e.g., albiglutide, dulaglutide, efpeglenatide, exenatide, liraglutide, lixisenatide, semaglutide, taspoglutide, tirzepatide), a sodium-glucose transporter (SGLT) 2 inhibitor (e.g. canagliflozin, dapagliflozin, empagliflozin, ertugliflozin, ipragliflozin, luseogliflozin, remogliflozin, sergliflozin, sotagliflozin, tofogliflozin), a sulfonylurea (e.g., acetohexamide, carbutamide, chlorpropamide, glibenclamide, glibornuride, gliclazide, glimepiride, glipizide, gliquidone, glisoxepide, glyclopyramide, glycyclamide, metahexamide, tolazamide, tolbutamide), a thiazolidinedione (e.g., AS-605240, balaglitazone, ciglitazone, darglitazone, englitazone, lobeglitazone, netoglitazone, pioglitazone, rivoglitazone, rosiglitazone, troglitazone), or a combination thereof.

In some embodiments, the pharmaceutical agent is or comprises an agent for treating infectious disease, including but not limited to, an antibiotic, an antiviral, an antifungal, an antiparasitic, or a combination thereof. Examples of antibiotics include, but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, methicillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilamide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin. Examples of antivirals include, but are not limited to, vidarabine, acyclovir, ganciclovir, valganciclovir, valacyclovir, cidofovir, famciclovir, ribavirin, amantadine, rimantadine, interferon, oseltamivir, palivizumab, rimantadine, zanamivir, nucleoside-analog reverse transcriptase inhibitors (NRTI, such as zidovudine, didanosine, zalcitabine, stavudine, lamivudine, and abacavir), non-nucleoside reverse transcriptase inhibitors (NNRTI, such as nevirapine, delavirdine and efavirenz), and protease inhibitors (such as saquinavir, ritonavir, indinavir, nelfinavir, amprenavir). Examples of antifungals include, but are not limited to, griseofulvin, ketoconazole, itraconizole, amphotericin B, nystatin, candicidin, parabens, chlorobutanol, phenol, sorbic acid, and thimerosal. Examples of antiparasitic agents include, but are not limited to, niclosamide, oxyclozanide, rafoxanide, closantel, dibromsalan, metabromsalan, tribromsalan, and nitazoxanide.

In some embodiments, the pharmaceutical agent is or comprises an agent or compound for treating neurodegenerative diseases (e.g., Alzheimer's disease, Parkinson's disease), such as aducanumab, a cholinesterase inhibitor, a glutamate regulator, an orexin receptor antagonist, levodopa, entacapone, tolcapone, opicapone, pramipexole, ropinirole, apomorphine, rotigotine, selegiline, rasagiline, safinamide, amantadine, istradefylline, trihexyphenidyl, benztropine, memantine, or a combination thereof. Examples of anti-Parkinson's drugs include, but are not limited to, dopamine replacement therapy (e.g. L-DOPA, carbidopa, COMT inhibitors such as entacapone or tolcapone), dopamine agonists (e.g. D1 agonists, D2 agonists, mixed D1/D2 agonists, bromocriptine, pergolide, cabergoline, ropinirole, pramipexole, piribedil, or apomorphine in combination with domperidone), histamine H2 antagonists, monoamine oxidase inhibitors (such as selegiline, rasagiline, safinamide and tranylcypromine), certain atypical antipsychotics such as pimavanserin (a non-dopaminergic atypical antipsychotic and inverse agonist of the serotonin 5-HT2A receptor), and amantadine. Examples of anti-Alzheimer's drugs include beta-secretase inhibitors, gamma-secretase inhibitors, cholinesterase inhibitors such as donepezil, galantamine or rivastigmine, HMG-CoA reductase inhibitors, NSAID's including ibuprofen, vitamin E, and anti-amyloid antibodies.

In some embodiments, the pharmaceutical agent is or comprises an agent or compound for treating a respiratory disease, including but not limited to, a bronchodilator (e.g., a beta2-adrenergic agonist (such as abediterol, albuterol, arformoterol, bambuterol, bitolterol, carmoterol, clenbuterol, fenoterol, formoterol, indacaterol, isoprenaline, isoxsuprine, levosalbutamol, mabuterol, olodaterol, orciprenaline, pirbuterol, procaterol, ritodrine, salbutamol, salmeterol, terbutaline, vilanterol, zilpaterol), an anticholinergics (such as atropine, benztropine, biperiden, bupropion, chlorpheniramine, clozapine, dextromethorphan, dicyclomine, dimenhydrinate, diphenhydramine, doxacurium, doxepin, doxylamine, flavoxate, glycopyrrolate, glycopyrronium, hexamethonium, hyoscyamine, ipratropium, mecamylamine, orphenadrine, oxitropium, oxybutynin, paroxetine, promethazine, propantheline bromide, quetiapine, scopolamine, solifenacin, tiotropium, tolterodine, trihexyphenidyl, tropicamide, tubocurarine, umeclidinium), a theophylline), a corticosteroid, a mast cell stabilizer (e.g., azelastine, cromoglicic acid, desloratadine, ketotifen, loratadine, mepolizumab, methylxanthines, nedocromil, olopatadine, omalizumab, palmitoylethanolamide, pemirolast, quercetin, rupatadine, tranilast, Vitamin D), a leukotriene receptor antagonist (e.g., montelukast, zafirlukast), an antihistamine (e.g., cetirizine, diphenhydramine, fexofenadine, loratadine), a respiratory stimulant (e.g., bicuculline, caffeine, doxapram, pentylenetetrazol, picrotoxin), a pulmonary surfactant (e.g., phospholipids such as dipalmitoylphosphatidylcholine (DPPC), surfactant proteins such as SP-A, SP-B, SP-C and SP-D), an antimicrobial, an antiviral, or a combination thereof.

In some embodiments, the pharmaceutical agent is or comprises a hormone, such as a steroid and derivatives thereof. Examples of hormones include, but are not limited to, incretins and incretin mimetics (such as GLP-1 and exenatide); androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones, such as levothyroxine sodium.

In some embodiments, the pharmaceutical agent is or comprises an antibody, such as a polyclonal antibody or a monoclonal antibody. The antibodies, as used herein, include full-length antibodies, fragments of an antibody (such as an antigen-binding fragment of a monoclonal antibody, Fab, Fab′, F(ab′)2, and Fv fragments), single chain Fv (scFv) mutants, multi-specific antibodies (such as bispecific antibodies generated from at least two intact antibodies), chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. Different classes of immunoglobulins can have different subunit structures and three-dimensional configurations. Antibodies can be naked or conjugated to other molecules such as toxins, radioisotopes, etc. Examples of antibodies include, but are not limited to, RITUXAN® (rituximab); REMICADE® (infliximab); HERCEPTIN® (trastuzumab); HUMIRA® (adalimumab); XOLAIR® (omalizumab); BEXXAR® (tositumomab); RAPTIVA® (efalizumab); ERBITUX® (cetuximab).

In some embodiments, the pharmaceutical agent is or comprises a therapeutic peptide. The therapeutic peptide can be, be about, be at least, be at least about, be at most, or be at most about 2, 3, 4, 5, 6, 7, 8, 9, 10, 15, 20, 25, 30, 35, 40, 50, 60, 70, 80, 90, or 100 amino acids in length, or a number or a range between any two of these values. In some embodiments, the therapeutic peptide is 2-40 amino acids in length. The therapeutic peptide can comprise peptide conjugates, such as peptides conjugated to polyethylene glycol (PEG), lipids, or proteins (e.g., Fc fragments). The therapeutic peptide can target, as non-limiting examples, G-protein coupled receptors (GPCRs), gonadotropin-releasing hormone (GnRH) receptor, glucagon-like peptide 1 (GLP-1) receptor, natriuretic peptide receptors, cytokine receptors, ion channels, AVP1 receptor, OT receptor, calcitonin receptor, glucagon receptor, erythropoietin (EPO) receptor, somatostatin receptor type 2 (SST 2), thrombin, glycoprotein IIb/IIIa, guanylyl cyclase-C (GC-C) receptor, calcium sensing receptor (CaSR), structural proteins, and adhesion molecules of the cell. Examples of therapeutic peptides include, but are not limited to, insulin, calcitonin, oxytocin, vasopressin, octreotide, leuprorelin, eptifibatide, glucagon, bivalirudin, pramlintide, exenatide, peginesatide. linaclotide, and etelcalcetide.

In some embodiments, the pharmaceutical agent is or comprises a therapeutic nucleic acid. The therapeutic nucleic acid can be, be about, be at least, be at least about, be at most, or be at most about 15, 20, 25, 30, 35, 40, 45, 50, 55, 60, 65, 70, 85, 90, 95, 100, 150, 200, 250, 300, 400, 500, 600, 700, 800, 900, or 1000 nucleotides in length, or a number or a range between any two of these values. The therapeutic nucleic acid or polynucleotide can be an inhibitory nucleic acid that can reduce the expression or translation of a gene or promote degradation of particular RNA species. In some embodiments, the therapeutic nucleic acid causes or promotes the transcription or activation of a gene or gene product. The therapeutic nucleic acid can comprise a promoter operably linked to a polynucleotide that encodes a protein. The therapeutic nucleic acid can encode an enhancer. In some embodiments, the therapeutic nucleic acid is a therapeutic RNA. Examples of an inhibitory nucleic acid include, but are not limited to, molecules targeted to an nucleic acid sequence, such as an small interfering RNA (siRNA), short hairpin RNA (shRNA), double-stranded RNA, micro RNA (miRNA), antisense oligonucleotide, ribozyme, and molecules targeted to a gene or gene product such as a nucleic acid aptamer.

### Barcode Molecules

Barcode molecules (e.g., barcode molecules attached to particles) can be partitioned, for example, in microwells or wells. The term “barcode” as used herein can be a verb or a noun. When used as a noun, the term “barcode” or “barcode molecule” refers to a label that can be attached to a polynucleotide, or any variant thereof, to convey information about the polynucleotide. For example, a barcode can be a polynucleotide sequence attached to fragments of the target nucleic acids associated with a cell in the partition. The barcode can then be sequenced alone or with the fragments of the target nucleic acids associated with the cell. The presence of the same barcode on multiple sequences or different barcodes on different sequences can provide information about the cell origin and/or the molecular origin of the sequences. When used as a verb, the term “barcode” refers to a process of attaching a barcode or a barcode molecule to a target nucleic acid associated with the cell.

Barcode molecules can be generated from a variety of different formats, including pre-designed polynucleotide barcodes, randomly synthesized barcode sequences, microarray-based barcode synthesis, random N-mers, or combinations thereof as will be understood by a person skilled in the art.

In some embodiments, the plurality of barcode molecules comprise, comprise about, comprise at least, comprise at least about, comprise at most, or comprise at most about 1, 5, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 7000000, 8000000, 9000000, 10000000, 20000000, 50000000, 100000000, 200000000, 300000000, 400000000, 500000000, 600000000, 700000000, 800000000, 900000000, 1000000000, or a number or a range between any two of these values.

A barcode molecule (or a segment of a barcode molecule, such as a partition barcode sequence or a molecular barcode sequence) can be in any suitable length. For example, a barcode molecule (or a segment of a barcode molecule) can be about 2 to about 500 nucleotides in length, about 2 to about 100 nucleotides in length, about 2 to about 50 nucleotides in length, about 2 to about 40 nucleotides in length, about 4 to about 20 nucleotides in length, or about 6 to 16 nucleotides in length. In some embodiments, the barcode molecule (or a segment of a barcode molecule) can be, be about, be at least, be at least about, be at most, or be at most about 2, 3, 4, 5, 6, 7, 8, 9, 10, 11, 12, 13, 14, 15, 16, 17, 18, 19, 20, 25, 30, 35, 40, 45, 50, 55, 60, 65, 70, 85, 90, 95, 100, 150, 200, 250, 300, 400, or 500 nucleotides in length, or a number or a range between any two of these values.

The barcode molecules used herein can comprise a partition barcode sequence and a molecular barcode sequence (e.g. a unique molecular identifier (UMI)). A barcode molecule can also comprise additional sequences, such as a target binding sequence or region capable of hybridizing to target nucleic acids (e.g. poly(dT) sequence), other recognition or binding sequences, a template switching oligonucleotide (e.g., GGG, such as rGrGrG), and primer sequences (e.g. sequencing primer sequence, such as Read 1 or a PCR primer sequence) for subsequent processing (e.g. PCR amplification) and/or sequencing.

The configuration of the various sequences comprised in a barcode molecule (e.g. partition barcode sequence, UMI, primer sequence, target binding sequence or region, and/or any additional sequences) can vary depending on, for example, the particular configuration desired and/or the order in which the various components of the sequence are added as will be understood to a person skilled in the art. In some embodiments, a barcode molecule has a configuration of 5′-primer sequence-partition barcode sequence-UMI-target binding sequence-3′. In some embodiments, a barcode molecule has a configuration of 5′-primer sequence-partition barcode sequence-UMI-template switching oligonucleotide-3′.

Partition Barcode Sequence

In some embodiments, the barcode molecules can comprise a partition barcode sequence. Partition barcode sequences can be used to identify the barcoded nucleic acids originate from the cell. (or the same partition). Barcoded nucleic acids that originate from the cell (or the same partition) can have an identical partition barcode sequence. A partition barcode sequence can be referred to as a partition specific barcode, such as a microwell specific barcode, a well position specific barcode, a well barcode, or a sample barcode. The partition barcode sequence of the barcode molecules in a partition can be identical or different.

In some embodiments, the partition barcode sequences can serve to track the target nucleic acids associated with the cell throughout the processing (e.g., location of the cells in a plurality of partitions, such as microwells or wells) when the partition barcode sequence associated with the target nucleic acids is determined during sequencing.

The number (or percentage) of barcode molecules introduced in a partition with partition barcode sequences having an identical sequence can be different in different embodiments. In some embodiments, the number of barcode molecules introduced in a partition with partition barcode sequences having an identical sequence is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode molecules introduced in a partition with partition barcode sequences having an identical sequence is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the partition barcode sequences of at least two barcode molecules introduced in a partition comprise an identical sequence. In some embodiments, at least two of the partition barcode sequences of the plurality of barcode molecules in the same partition are identical.

A partition barcode sequence can be unique (or substantially unique) to a partition. The number of unique partition barcode sequences can be different in different embodiments. In some embodiments, the number of unique partition barcode sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of unique partition barcode sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values, of the partition barcode sequences of the barcode molecules introduced in a partition. For example, the partition barcode sequences of barcode molecules introduced in two partitions can comprise different sequences. In some embodiments, the partition barcode sequences of at least one barcode molecules in at least two different partitions are different.

In some embodiments, barcode molecules are introduced to the plurality of partitions such that different sets of a plurality of barcode molecules introduced in different partitions have different partition barcode sequences and a same set of plurality of barcode molecules introduced in a same partition have same partition barcode sequence. For example, target nucleic acids associated with a cell in a partition will be barcoded with the same partition barcode sequences.

The length of a partition barcode sequence of a barcode molecule (or a partition barcode sequence of each barcode molecule or all partition barcode sequences of the plurality of barcode molecules) can be different in different embodiments. In some embodiments, a partition barcode sequence of a barcode molecule (or each partition barcode sequence of each barcode molecule or all partition barcode sequences of the plurality of barcode molecules) is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

Molecular Barcode Sequence

In some embodiments, a barcode molecular can comprise a molecular barcode sequence or molecular label. Molecular barcode sequences can be unique molecule identifiers (UMIs). Molecular barcode sequences can be used to identify molecular origins of the barcoded nucleic acids. Molecular barcode sequences (e.g., UMIs) are short sequences used to uniquely tag each molecule in a sample in some embodiments. The molecular barcode sequences of the barcode molecules partitioned into a partition can be identical or different.

In some embodiments, the molecular barcode sequences of the plurality of barcode molecules are different. The number (or percentage) of molecular barcode sequences of barcode molecules introduced in a partition (e.g., a microwell or a well) with different sequences can be different in different embodiments. In some embodiments, the number of molecular barcode sequences of barcode molecules introduced in a partition with different sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of molecular barcode sequences of barcode molecules introduced in a partition with different sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the molecular barcode sequences of two barcode molecules of the plurality of barcode molecules introduced in a partition can comprise different sequences.

The number of barcode molecules introduced in a partition with molecular barcode sequences having an identical sequence can be different in different embodiments. In some embodiments, the number of barcode molecules introduced in a partition with molecular barcode sequences having an identical sequence is, is about, is at least, is at least about, is at most, or is at most about, 1, 2, 3, 4, 5, 6, 7, 8, 9, 10, 20, 30, 40, 50, 60, 70, 80, 90, 100, or a number or a range between any two of these values. For example, the molecular barcode sequences of two barcode molecules introduced in a partition can comprise an identical sequence.

The number of unique molecular barcode sequences can vary. For example, the number of unique molecular barcode sequences can be, be about, be at least, be at least about, be at most, or be at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

In some embodiments, at least two of the molecular barcode sequences of the plurality of barcode molecules in a partition comprise different molecular barcode sequences (e.g., unique molecular identifiers).

The length of a molecular barcode sequence of a barcode molecule (or a molecular barcode sequence of each barcode molecule) can be different in different embodiments. In some embodiments, a molecular barcode sequence of a barcode molecule (or a molecular barcode sequence of each barcode molecule) is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

In some embodiments, a barcode molecule can comprise a primer sequence. The primer sequence can be a sequencing primer sequence (or a sequencing primer binding sequence) or a PCR primer sequence (or PCR primer binding sequence). For example, the sequencing primer can be a Read 1 sequence. In some embodiments, the barcode molecule comprise a PCR primer binding sequence, which allows for PCR amplification of a barcoded nucleic acid (FIG. 1D).

The length of the primer sequence can vary. In some embodiments, the primer sequence is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. The number (or percentage) of barcode molecules in a partition (e.g., a microwell or a well) each comprising a primer sequence (or each comprising an identical primer sequence) can be different in different embodiments. In some embodiments, the number of barcode molecules in a partition (e.g., a microwell or a well) each comprising a primer sequence (such as a PCR primer binding sequence) is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode molecules in a partition (e.g., a microwell or a well) each comprising a primer sequence (or each comprising an identical primer sequence) is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

In some embodiments, each of the plurality of barcode molecules comprises a primer sequence (e.g., a sequencing primer sequence, including but not limited to, a Read 1 sequence, a Read 2 sequence, or a portion thereof).

In some embodiments, a barcode molecule comprises a target binding sequence or region capable of hybridizing to the target nucleic acids, a particular type of target nucleic acids (e.g. mRNA), and/or specific target nucleic acids (e.g. specific gene of interest). In some embodiments, the target binding sequence comprises a poly(dT) sequence and/or a sequence capable of hybridizing to the plurality of target nucleic acids.

The length of a target binding sequence can vary. For example, the target binding sequence can be, be about, be at least, be at least about, be at most, or be at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. The target binding sequence can be 12-18 deoxythymidines in length. In some embodiments, the target binding sequence can be 20 nucleotides or longer to enable their annealing in reverse transcription reactions at higher temperatures as will be understood by a person of skill in the art.

In some embodiments, barcode molecules comprising target binding sequences are introduced into the partitions together with other reagents such as the reverse transcription reagents. The number of the barcode molecules introduced into a partition comprising a target binding sequence can vary. For example, the number of barcode molecules introduced into a partition comprising a target binding sequence (e.g., poly(dT) sequence) can be, be about, be at least, be at least about, be at most, or be at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, or a number or a range between any two of these values.

In some embodiments, the target binding sequence can be on a 3′ end of a barcode molecule of the plurality of barcode molecules introduced in a partition. Barcode molecules each comprising a poly(dT) target binding sequence can be used to capture (e.g., hybridize to) 3′ end of polyadenylated mRNA transcripts in a target nucleic acid for a downstream 3′ gene expression library construction.

In some embodiments, the target binding sequence can comprise a poly(dT) sequence which is a single-stranded sequence of deoxythymidine (dT) used for first-strand cDNA synthesis catalyzed by reverse transcriptase. In some embodiments, the target binding sequence comprises a poly(dT) sequence can be introduced into the partitions as extension primers to synthesize the first-strand cDNA using the target nucleic acid (e.g. RNA) as a template.

In some embodiments, the poly(dT) of the barcode molecules introduced into a partition are identical (e.g., same number of dTs). In some embodiments, the poly(dT) of the barcode molecules introduced into a partition are different (e.g. different numbers of dTs). The percentage of the barcode molecules of the plurality of barcode molecules introduced into a partition with an identical poly(dT) sequence can vary. In some embodiments, the percentage of the barcode molecules of the plurality of barcode molecules introduced into a partition with an identical poly(dT) sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

In some embodiments, the target binding regions of all barcode molecules of the plurality of barcode molecules comprise poly(dT) capable of hybridizing to poly(A) tails of mRNA molecules (or poly(dA) regions or tails of DNA). In some embodiments, the target binding regions of some barcode molecules of the plurality of barcode molecules comprise gene-specific or target-specific primer sequences. For example, a barcode molecule of the plurality of barcode molecules can also comprise a target binding region capable of hybridizing to a specific target nucleic acid associated with the cell, thereby capturing specific targets or analytes of interest. For example, the target binding region capable of hybridizing to a specific target nucleic acid can be a gene-specific primer sequence. The gene-specific primer sequences can be designed based on known sequences of a target nucleic acid of interest. The gene-specific primer sequences can span a nucleic acid region of interest, or adjacent (upstream or downstream) of a nucleic acid region of interest.

The length of the gene-specific primer sequence can vary. For example, a gene-specific primer sequence can be, be about, be at least, be at least about, be at most, or be at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. In some embodiments, the gene-specific primer sequence is at least 10 nucleotides in length.

The number of the barcode molecules introduced into a partition comprising a gene-specific primer sequence can vary. For example, the number of barcode molecules introduced into a partition comprising a gene-specific primer sequence can be, be about, be at least, be at least about, be at most, or be at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the barcode molecules introduced into a partition comprises a set of different gene-specific primer sequences each capable of binding to a specific target nucleic acid sequence.

The number of different gene-specific primer sequences of the barcode molecules introduced into a partition can vary. For example, the number of different gene-specific primer sequences of the barcode molecules introduced into a partition can be, be about, be at least, be at least about, be at most, or be at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values.

The number of target nucleic acids of interest (e.g. genes of interest) that the barcode molecules introduced into a partition are capable of binding can vary. For example, the number of target nucleic acids of interest (e.g. genes of interest) the barcode molecules introduced into a partition are capable of binding can be, be about, be at least, be at least about, be at most, or be at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values. In some embodiments, one barcode molecule introduced into a partition can bind to a molecule (or a copy) of a target nucleic acid. Barcode molecules introduced into a partition can bind to molecules (or copies) of a target nucleic acid or a plurality of target nucleic acids.

In some embodiments, the barcode molecules of the plurality of barcode molecules each comprise a poly(dT) sequence, a gene-specific primer sequence, and/or both. The poly(dT) sequence and the gene-specific primer sequence can be on a same barcode molecule or different barcode molecules of the plurality of barcode molecules introduced into a partition.

The ratio of the number of barcode molecules introduced into a partition comprising a poly(dT) sequence and the number of barcode molecules introduced into a partition comprising a gene-specific primer sequence can vary. For example, the ratio can be, be about, be at least, be at least about, be at most, be at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values.

In some embodiments, a barcode molecule (or each barcode molecule of the plurality of barcode molecules) comprises a template switching oligonucleotide (TSO). A primer comprising a target binding region, such as a poly(dT) sequence, can hybridize to a target nucleic acid (e.g., an mRNA) and be extended by, for example, reverse transcription to generate an extended primer comprising a reverse complement of the target nucleic acid, or a portion thereof (e.g., cDNA). The extended primer or cDNA can be further extended to include the reverse complement of a TSO oligonucleotide or barcode molecule. The resulting barcoded nucleic acid includes the barcodes of the barcode molecule on the 3′-end.

In some embodiments, a barcode molecule does not comprise a TSO. A barcode molecule comprising a target binding region, such as a poly(dT) sequence, can hybridize to a target nucleic acid (e.g., an mRNA) and be extended by, for example, reverse transcription to generate an extended primer comprising a reverse complement of the target nucleic acid, or a portion thereof (e.g., cDNA). The extended primer or cDNA can be further extended to include the reverse complement of a template switching oligonucleotide. The resulting barcoded nucleic acid includes the barcodes of the barcode molecule on the 5′-end (FIG. 1D). The resulting barcoded nucleic acid (e.g., extended cDNA) can comprise a PCR primer binding sequence introduced in the reverse complement of the template switching oligonucleotide.

A TSO is an oligonucleotide that hybridizes to untemplated C nucleotides added by a reverse transcriptase during reverse transcription. The TSO can hybridize to the 3′ end of a cDNA molecule. The TSO can include one or more nucleotides with guanine (G) bases on the 3′-end of the TSO, with which the one or more cytosine (C) bases added by a reverse transcriptase to the 3′-end of a cDNA can hybridize. The series of G bases can comprise 1 G base, 2 G bases, 3 G bases, 4 G bases, 5 G bases or more than 5 G bases. The series of G bases can be ribonucleotides. The reverse transcriptase can further extend the cDNA using the TSO as the template to generate a barcoded cDNA comprising the TSO. The length of a TSO can vary. For example, a TSO can be, be about, be at least, be at least about, be at most, or be at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

The number of the barcode molecules introduced into a partition comprising a TSO can vary. In some embodiments, the number of barcode molecules introduced into a partition comprising a TSO is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

The TSO of the barcode molecules introduced into a partition can be identical. In some embodiments, the TSO of the barcode molecules introduced into a partition is different. The percentage of the barcode molecules of the plurality of barcode molecules introduced into a partition with an identical TSO sequence can be different in different embodiments. In some embodiments, the percentage of the barcode molecules of the plurality of barcode molecules introduced into a partition with an identical TSO sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

### Barcoding Target Nucleic Acids

The method described herein can comprise barcoding target nucleic acids associated with a cell in the partition (e.g., microwell or well) using the barcode molecules to generate a barcoded nucleic acids (e.g., target nucleic acids each hybridized with a barcode molecule, single-stranded barcoded nucleic acids, or double-stranded barcoded nucleic acids).

The method can, in some embodiments, further comprises releasing the plurality of target nucleic acids associated with the one or more cells in the partition prior to barcoding the plurality of target nucleic acids. In some embodiments, releasing the plurality of target nucleic acids associated with the one or more cells comprises lysing the plurality of cells. For example, prior to barcoding the target nucleic acids, the method can comprise lysing the cells to release the content of the cells within the partition. Lysis agents can be contacted with the cells or cell suspension concurrently. Lysis agents can be introduced to the cells prior to or immediately after subjecting the cells to various pharmaceutical agents in the partitions (e.g., microwells or wells). In some embodiments, the lysis agent does not interfere with effect of the pharmaceutical agent on the cells. Non-limiting examples of lysis agents include bioactive reagents, such as lysis enzymes, or surfactant based lysis solutions including non-ionic surfactants (e.g., Triton X-100 and Tween 20) and ionic surfactants (e.g., sodium dodecyl sulfate (SDS)). Lysis methods including, but not limited to, thermal, acoustic, electrical, or mechanical cellular disruption can also be used.

The barcode molecules can be introduced to the cells prior to or after subjecting the cells to a pharmaceutical agent. In some embodiments, the barcode molecules do not interfere with effect of the pharmaceutical agent on the cells. In some embodiments, the present method comprises introducing the plurality of the barcode molecules into the partition prior to subjecting the one or more cells in the partition with the pharmaceutical agent. In some embodiments, the present method comprises introducing the plurality of the barcode molecules into the partition after subjecting the one or more cells in the partition with the pharmaceutical agent.

First Strand Synthesis and Single-Stranded Barcoded Nucleic Acids

In some embodiments, barcoding the plurality of target nucleic acids comprises a reverse transcription reaction, for example, to generate a plurality of barcoded nucleic acids comprising cDNAs. In some embodiments, barcoding the plurality of target nucleic acids comprises extending the plurality of barcode molecules using the plurality of target nucleic acids as templates to generate the plurality of barcoded nucleic acids comprising a plurality of single-stranded barcoded nucleic acids. In some embodiments, the plurality of single-stranded barcoded nucleic acids can be hybridized to the plurality of target nucleic acids in the partition.

In some embodiments, barcoding target nucleic acids associated with a cell in the partition can comprise extending the barcode molecules using the target nucleic acids as templates to generate partially single-stranded/partially double-stranded barcoded nucleic acids hybridized to the target nucleic acids in the partition (or after target nucleic acids hybridized with barcode molecules are pooled). The partially single-stranded/partially double-stranded barcoded nucleic acids hybridized to target nucleic acids can be separated by denaturation (e.g., heat denaturation or chemical denaturation using for example, sodium hydroxide) to generate single-stranded barcoded nucleic acids of the plurality of barcoded nucleic acids. The single-stranded barcoded nucleic acids can comprise a barcode molecule and an oligonucleotide complementary to the target nucleic acids. In some embodiments, the single-stranded barcoded nucleic acids are generated by reverse transcription using a reverse transcriptase. In some embodiments, the single-stranded barcoded nucleic acids is generated by using a DNA polymerase.

In some embodiments, the method further comprises introducing a plurality of TSO into the partition. Barcoding the plurality of target nucleic acids can comprise extending the plurality of barcode molecules using the plurality of target nucleic acids and the plurality of template switching oligonucleotides as templates to generate the plurality of barcoded nucleic acids comprising a plurality of single-stranded barcoded nucleic acids.

For example, the single-stranded barcoded nucleic acids can be cDNA produced by extending a barcode molecule using a target RNA associated with the cell as a template. The single-stranded barcoded nucleic acids can be further extended using a TSO. The TSO can be introduced into the partitions together with the reverse transcription reagents. For example, a reverse transcriptase can be used to generate a cDNA by extending a barcode molecule hybridized to an RNA. After extending the barcode molecule to the 5′-end of the RNA, the reverse transcriptase can add one or more nucleotides with cytosine (C) bases (e.g. two or three) to the 3′-end of the cDNA. The TSO can include one or more nucleotides with guanine (G) bases (e.g. two or more) on the 3′-end of the TSO. The nucleotides with G bases can be ribonucleotides. The G bases at the 3′-end of the TSO can hybridize to the cytosine bases at the 3′-end of the cDNA. The reverse transcriptase can further extend the cDNA using the TSO as the template to generate a cDNA with the reverse complement of the TSO sequence on its 3′-end. The barcoded nucleic acid can include the barcode sequences (e.g., partition barcode sequence and molecular barcode sequence (e.g., UMI)) on the 5′-end and a TSO sequence at its 3′-end (FIG. 1D).

In some embodiments, barcoding the target nucleic acids comprises extending the barcode molecules using the target nucleic acids as templates and the barcode molecules as TSO to generate single-stranded barcoded nucleic acids that are hybridized to the target nucleic acids. In some embodiments, the present method further comprises introducing a plurality of extension primers to the partition. Barcoding the plurality of target nucleic acids can comprise extending the plurality of extension primers using the plurality of target nucleic acids as templates and the plurality of barcode molecules as template switching oligonucleotides to generate the plurality of barcoded nucleic acids comprising a plurality of single-stranded barcoded nucleic acids.

In some embodiments, the barcode molecules are not attached to a particle and the barcode molecules can comprise TSO. Extension primers (e.g. oligonucleotides comprising a poly(dT) sequence) can be introduced into the partitions which hybridize to a target nucleic acid (e.g. the poly-adenylated mRNA). The extension primers can be extended using the target nucleic acids as a template. For example, a reverse transcriptase can be used to generate a cDNA by extending an extension primer hybridized to an RNA. After extending the extension primers to the 5′-end of the RNA, the reverse transcriptase can add one or more C bases (e.g. two or three) to the 3′-end of the cDNA. The TSO or barcode molecule can include one or more G bases (e.g. two or more) on the 3′-end of the TSO. The nucleotides with guanine bases can be ribonucleotides. The G bases at the 3′-end of the TSO or barcode molecule can hybridize to the cytosine bases at the 3′-end of the cDNA. The reverse transcriptase can switch template from the mRNA to the TSO or barcode molecule. The reverse transcriptase can further extend the cDNA using the TSO or barcode molecule as the template to generate a cDNA further comprising the reverse complement of the TSO or barcode molecule. In this case, the barcode sequences (e.g., partition barcode sequence and molecular barcode sequence (e.g., UMI)) are on the 3′-end of the generated cDNA.

In some embodiments, each of the plurality of single-stranded barcoded nucleic acids is hybridized to one of the plurality of target nucleic acids and one of the plurality of template switching oligonucleotides in the partition.

The single-stranded barcoded nucleic acids can be separated from the template target nucleic acids by digesting the template target nucleic acids (e.g., using RNase), by chemical treatment (e.g., using sodium hydroxide), by hydrolyzing the template target nucleic acids, or via a denaturation or melting process by increasing the temperature, adding organic solvents, or increasing pH. Following the melting process, the target nucleic acids can be removed (e.g. washed away) and the single-stranded barcoded nucleic acids can be retained in the partition (e.g. through attachment to the partitions or through attachments to particles which can be retained in the partitions). In some embodiments, the method further comprises removing the plurality of target nucleic acids and the plurality of template switching oligonucleotides hybridized to the single-stranded barcoded nucleic acids. In some embodiments, removing the plurality of target nucleic acids comprises denaturation, thermal denaturation, digesting, or hydrolyzing the plurality of target nucleic acids.

In some embodiments, each of the plurality of single-stranded barcoded nucleic acid comprises a sequence of a barcode molecule of the plurality of barcode molecules (e.g., an actual sequence of the barcode molecule), a sequence of a target nucleic acid of the plurality of target nucleic acids (e.g. a reverse complement of the target nucleic acid), and/or a sequence of an extension primer of the plurality of extension primers (e.g., an actual sequence of the extension primer).

Second Strand Synthesis, Amplification, and Double-Stranded Barcoded Nucleic Acids

The method can further comprise amplifying the plurality of barcoded nucleic acids to generate a plurality of double-stranded barcoded nucleic acids in the partition using the single-stranded barcoded nucleic acids as templates. The amplifying step can be used to amplify the product of first strand synthesis and/or RT reaction as described here.

For example, barcoding target nucleic acids associated with the cell in the partition (e.g., microwell or well) can comprise amplifying the barcoded nucleic acids (such as a single-stranded barcoded nucleic acid, or a cDNA generated by using a barcode molecule as disclosed herein). The amplification can comprise generating barcoded nucleic acids comprising double-stranded barcoded nucleic acids in the partition using the single-stranded barcoded nucleic acids as templates. The double-stranded barcoded nucleic acids can be generated from the single-stranded barcoded nucleic acids retained in the partition using, for example, second-strand synthesis or one-cycle PCR. Amplification of the barcoded nucleic acids can include additional cycles of PCR reactions.

The generated double-stranded barcoded nucleic acid can be denaturized or melted to generate two single-stranded barcoded nucleic acids: one single-stranded barcoded nucleic acid retained in the partition (e.g., attached to the particle) and the other single-stranded barcoded nucleic acid released into the solution from the retained single-stranded barcoded nucleic acid that can then be pooled to provide a pooled mixture outside the partitions. Both single-stranded barcoded nucleic acids (e.g. retained in the partitions or pooled outside the partitions) have a sequence comprising a sequence of a barcode molecule (e.g. partition barcode sequence and molecular barcode sequence (e.g., UMI)) and a sequence of a target nucleic acid or a reverse complement thereof.

In some embodiments, amplifying the plurality of barcoded nucleic acids comprises amplifying the plurality of barcoded nucleic acids in the partition to generate the plurality of double-stranded barcoded nucleic acids. The plurality of target nucleic acids in a partition can be barcoded and the plurality of barcoded nucleic acids generated are then amplified in the same partition. Further, the plurality of target nucleic acids in a partition can be barcoded and the plurality of barcoded nucleic acids generated are then amplified in the same reaction. For example, the reaction can be a one-step RT-PCR reaction.

Each of the plurality of barcode molecules can comprise a primer sequence. In some embodiments, the primer sequence can comprise a PCR primer sequence. Amplifying the plurality of barcoded nucleic acids can comprise amplifying the plurality of barcoded nucleic acids using the primer sequences in single-stranded barcoded nucleic acids of the plurality of single-stranded barcoded nucleic acids, or products thereof.

In some embodiments, the barcoding process comprises reverse transcription using an mRNA associated with the cell (and optionally a TSO) as template to generate a barcoded cDNA molecule (optionally with a reverse complement of a TSO) and amplification of the barcoded cDNA by PCR. In some embodiments, the reverse transcription (RT) and the amplification (by PCR) are performed in a one-step RT-PCR reaction.

For example, an enzyme mixture comprising a reverse transcriptase and a PCR polymerase (e.g., Taq DNA Polymerase) can be introduced to a cell in a partition, optionally after the cell is lysed. The reverse transcription can be carried out at a first temperature in the partition, and the PCR reaction can be carried out at a second temperature in the same partition. In some embodiments, the second temperature is higher than the first temperature. In some embodiments, the reverse transcriptase is inactivated at the second temperature.

The first temperature can, for example, be, be about, be at least, be at least about, be at most, or be at most about, 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 55° C., 60° C., 65° C., 70° C., 75° C., 80° C., or a number or a range between any two of these values. In some embodiments, the first temperature is about 35° C. to about 45° C. The reverse transcriptase can be inactivated at a temperature that is, is about, is at least, is at least about, is at most, is at most about, 55° C., 60° C., 65° C., 70° C., 75° C., 80° C., 85° C., 90° C., 95° C., or a number or a range between any two of these values. In some embodiments, the reverse transcription is carried out at a first temperature of about 42° C., optionally the reverse transcriptase is inactivated at a temperature of at least 70° C.

The second temperature can, for example, be, be about, be at least, be at least about, be at most, or be at most about, 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., or a number or a range between any two of these values. In some embodiments, the first temperature is about 65° C. to about 98° C., such as about 67° C., about 72° C., about 95° C., or about 98° C. In some embodiments, the PCR reaction is carried out at a temperature at which the reverse transcriptase is inactivated. In some embodiments, the reverse transcription is carried out at a first temperature of about 42° C., the reverse transcriptase is then inactivated at a temperature of about 95° C., and the PCR reaction is subsequently carried out at a second temperature of about 65° C. to about 98° C.

### Pooling

The methods disclosed herein can comprise pooling the plurality of barcoded nucleic acids, or products thereof, in each of the plurality of partitions to generate pooled barcoded nucleic acids. Subjecting the plurality of barcoded nucleic acids, or products thereof, to sequencing can comprise subjecting the pooled barcoded nucleic acids, or products thereof, to sequencing. In some embodiments, pooling the plurality of barcoded nucleic acids, or products thereof, comprises pooling the plurality of double-stranded barcoded nucleic acids in each of the plurality of partitions to generate the pooled barcoded nucleic acids. For example, the method can comprise pooling the barcoded nucleic acids after barcoding the target nucleic acids and before sequencing the barcoded nucleic acids to obtain pooled barcoded nucleic acids.

In some embodiments, pooling barcoded nucleic acids occurs after generating double-stranded barcoded nucleic acids (e.g., after second strand synthesis) or after generating amplified barcoded nucleic acids. The amplified barcoded nucleic acids can be subject to sequencing library construction prior to sequencing. In some embodiments, synthesis of single-stranded barcoded nucleic acids and double-stranded barcoded nucleic acids occur after the pooling of target nucleic acids hybridized with barcode molecules.

In some embodiments the barcode molecules are attached to particles, only single-stranded barcoded nucleic acids released into bulk (e.g., after amplification of the barcoded nucleic acids) are collected by pooling, and the particles are not pooled (e.g. not removed from the partitions) but retained in the partitions (e.g. by an external magnetic field applied on magnetic beads), thereby allowing one to trace the origin of the pooled barcoded nucleic acids, for example, to its original location in the partitions.

The pooled barcoded nucleic acids can be single-stranded or double-stranded (e.g. generated from the single-stranded pooled barcoded nucleic acids by PCR amplification). The pooled barcoded nucleic acids (e.g. amplified barcoded cDNA) can be purified, and optionally further amplified, prior to sequencing library construction. The pooled barcoded nucleic acids with desired length can be selected.

### Sequencing Library Construction

The barcoded nucleic acids (e.g. pooled barcoded nucleic acids) can be further processed prior to sequencing to generate processed barcoded nucleic acids. For example, the method herein can include amplification of barcoded nucleic acids, fragmentation of amplified barcoded nucleic acids, end repair of fragmented barcoded nucleic acids, A-tailing of fragmented barcoded nucleic acids that have been end-repaired (e.g., to facilitate ligation to adapters), and attaching (e.g. by ligation and/or PCR) with a second sequencing primer sequence (e.g. a Read 2 sequence), sample indexes (e.g. short sequences specific to a given sample library), and/or flow cell binding sequences (e.g. P5 and/or P7). Additional PCR amplification can also be performed. This process can also be referred to as sequencing library construction.

PCR amplification can be carried out to generate sufficient mass for the subsequent library construction processes. In some embodiments, the present method comprises performing a polymerase chain reaction in bulk on the pooled barcoded nucleic acids, or the fragmented barcoded nucleic acids, to generate amplified barcoded nucleic acids. For example, the method can comprise performing a polymerase chain reaction in bulk, subsequent to the pooling, on the pooled barcoded nucleic acids, thereby generating amplified barcoded nucleic acids. In some embodiments, performing the polymerase chain reaction in bulk is subsequent to fragmenting the pooled barcoded nucleic acids. The amplification for library preparation can be a separate process from the amplification of the first strand barcoded nucleic acid generated by, for example, the RT reaction as described herein (such as a one-step RT-PCR reaction).

In some embodiments, the method comprises fragmenting the pooled barcoded nucleic acids to generate fragmented barcoded nucleic acids to generate fragmented barcoded nucleic acids prior to subjecting the plurality of barcoded nucleic acids, or products thereof, to sequencing. For example, the method can comprise fragmenting (e.g., via enzymatic fragmentation, mechanical force, chemical treatment, etc.) the pooled barcoded nucleic acids to generate fragmented barcoded nucleic acids. Fragmentation can be carried out by any suitable process such as physical fragmentation, enzymatic fragmentation, or a combination of both. For example, the barcoded nucleic acids can be sheared physically using acoustics, nebulization, centrifugal force, needles, or hydrodynamics. The barcoded nucleic acids can also be fragmented using enzymes, such as restriction enzymes and endonucleases.

Fragmentation yields fragments of a desired size for subsequent sequencing. The desired sizes of the fragmented nucleic acids are determined by the limitations of the next generation sequencing instrumentation and by the specific sequencing application as will be understood by a person skilled in the art. For example, when using Illumina technology, the fragmented nucleic acids can have a length of between about 50 bases to about 1,500 bases. In some embodiments, the fragmented barcoded nucleic acids have about 100 bp to 700 bp in length.

Fragmented barcoded nucleic acids can undergo end-repair and A-tailing (to add one or more adenine bases) to form an A overhang. This A overhang allows adapter containing one or more thymine overhanging bases to base pair with the fragmented barcoded nucleic acids.

Fragmented barcoded nucleic acids can be further processed by adding additional sequences (e.g. adapters) for use in sequencing based on specific sequencing platforms. Adapters can be attached to the fragmented barcoded nucleic acids by ligation using a ligase and/or PCR. For example, fragmented barcoded nucleic acids can be processed by adding a second sequencing primer sequence. The second sequencing primer sequence can comprise a Read 2 sequence. An adapter comprising the second primer sequence can be ligated to the fragmented barcoded nucleic acids after, for example, end-repair and A tailing, using a ligase. The adaptor can include one or more thymine (T) bases that can hybridize to the one or more A bases added by A tailing. An adaptor can be, for example, partially double-stranded or double stranded. In some embodiments, the amplified barcoded nucleic acids comprise a sequencing primer sequence.

The adapter can also include platform-specific sequences for fragment recognition by specific sequencing instrument. In some embodiments, the amplified barcoded nucleic acids comprise a sequence for attaching the amplified barcoded nucleic acids to a flow well. For example, the amplified barcoded nucleic acids can comprise an adapter that comprises a sequence for attaching the fragmented barcoded nucleic acids to a flow well of Illumina platforms, such as a P5 sequence, a P7 sequence, or a portion thereof. Different adapter sequences can be used for different next generation sequencing instrument as will be understood by a person skilled in the art.

The adapter can also contain sample indexes to identify samples and to permit multiplexing. Sample indexes enable multiple samples to be sequenced together (i.e. multiplexed) on the same instrument flow cell as will be understood by a person skilled in the art. Adapters can comprise a single sample index or a dual sample indexes depending on the implementations such as the number of libraries combined and the level of accuracy desired.

In some embodiments, the amplified barcoded nucleic acids generated from sequencing library construction can include a P5 sequence, a sample index, a Read 1 sequence, a partition barcode sequence, a molecular barcode sequence (e.g., UMI), a poly(dT) sequence, a target biding region, a sequence of a target nucleic acid or a portion thereof, a Read 2 sequence, a sample index, and/or a P7 sequence (e.g., from 5′-end to 3′-end). In some embodiments, the amplified barcoded nucleic acids can include a P5 sequence, a sample index, a Read 1 sequence, a partition barcode sequence, a molecular barcode sequence (e.g., UMI), a sequence of a template switching oligonucleotide, a sequence of a target nucleic acid or a portion thereof, a Read 2 sequence, a sample index, and/or a P7 sequence (e.g., from 5′-end to 3′-end).

Sequencing the barcoded nucleic acids, or products thereof, can comprise sequencing products of the barcoded nucleic acids. Products of the barcoded nucleic acids can include the processed nucleic acids generated by any step of the sequencing library construction process, such as amplified barcoded nucleic acids, fragmented barcoded nucleic acids, fragmented barcoded nucleic acids comprising additional sequences such as the second sequencing primer sequence and/or adapter sequences described herein.

### Sequencing Barcoded Nucleic Acids

The method disclosed herein can comprise sequencing the barcoded nucleic acids or products thereof to obtain nucleic acid sequences of the barcoded nucleic acids. The barcoded nucleic acids generated by the method disclosed herein can comprise barcoded nucleic acids pooled, from each partition, into a pooled mixture outside the partitions. The barcoded nucleic acids retained in a partition and the pooled barcoded nucleic acids in a pooled mixture outside the partitions can be sequenced using a same or different sequencing techniques.

In some embodiments, sequencing the plurality of barcoded nucleic acids or products thereof comprises sequencing the pooled barcoded nucleic acids to obtain nucleic acid sequences of the pooled barcoded nucleic acids. As used herein, a “sequence” can refer to the sequence, a complementary sequence thereof (e.g., a reverse, a compliment, or a reverse complement), the full-length sequence, a subsequence, or a combination thereof. The nucleic acids sequences of the pooled barcoded nucleic acids can each comprise a sequence of a barcode molecule (e.g., the partition barcode sequence and the molecular barcode sequence (e.g., UMI)) and a sequence of a target nucleic acid associated with the cell or a reverse complement thereof.

Pooled barcoded nucleic acids can be sequenced using any suitable sequencing method identifiable. For example, sequencing the pooled barcoded nucleic acids can be performed using high-throughput sequencing, pyrosequencing, sequencing-by-synthesis, single-molecule sequencing, nanopore sequencing, sequencing-by-ligation, sequencing-by-hybridization, next generation sequencing, massively-parallel sequencing, primer walking, and any other sequencing methods known in the art and suitable for sequencing the barcoded nucleic acids generated using the methods herein described.

### Analysis

Method disclosed herein can comprise determining a profile of the cells, for example from the sequence of the barcode nucleic acids. The profile determined, in some embodiments, can be used to determine one or more effects of the pharmaceutical condition or the pharmaceutical agent on the cells. In some embodiments, the plurality of barcoded nucleic acids are analyzed to determine one or more effects of the pharmaceutical condition or the pharmaceutical agent on the cells.

The obtained nucleic acid sequences of the plurality of barcoded nucleic acids (e.g. nucleic acid sequences of pooled barcoded nucleic acids) can be subjected to any downstream post-sequencing data analysis as will be understood by a person of skill in the art. The sequence data can undergo a quality control process to remove adapter sequences, low-quality reads, uncalled bases, and/or to filter out contaminants. The high-quality data obtained from the quality control can be mapped or aligned to a reference genome or assembled de novo.

Profile analysis, for example gene expression quantification and differential expression analysis, can be carried out to identify genes whose expression differs in different cells. Barcoded nucleic acids from a cell can have an identical partition barcode sequence in the sequencing data and can be identified. Barcoded nucleic acids from different cells can have different partition barcode sequences in the sequencing data and can be identified. Barcoded nucleic acids with an identical partition barcode sequence, an identical target sequence, and different molecular barcode sequences in the sequencing data can be quantified and used to determine the expression of the target.

In some embodiments, the method can comprise determining a profile (e.g. an expression profile, a transcription profile, an omics profile, or a multi-omics profile) of the one or more cells from the sequences of the barcoded nucleic acids. In some embodiments, the profile comprises a single omics profile, such as a transcriptome profile. In some embodiments, the profile comprises a multi-omics profile, which can include profiles of genome (e.g. a genomics profile), proteome (e.g. a proteomics profile), transcriptome (e.g. a transcriptomics profile), epigenome (e.g. an epigenomics profile), metabolome (e.g. a metabolomics profile), and/or microbiome (e.g. microbiome profile). In some embodiments, the multi-omics profile comprises a genomics profile, a proteomics profile, a transcriptomics profile, an epigenomics profile, a metabolomics profile, a chromatics profile, a protein expression profile, a cytokine secretion profile, or a combination thereof. Differences/changes in profile among cells subjected to different pharmaceutical conditions (e.g., different concentrations of a pharmaceutical agent) are indicative of and can be used to analyze the effect of the pharmaceutical conditions on the cells, which can be used, for example, selecting compounds of desirable therapeutical effects.

In some embodiments, the profile comprises an expression of a target nucleic acid of the plurality of target nucleic acids. For example, the expression of the target nucleic acid can comprise an abundance of the target nucleic acid. The abundance of the target nucleic acid can comprise an abundance of molecules of the target nucleic acid barcoded using the barcode molecules. The abundance of the molecules of the target nucleic acid can comprise a number of occurrences of the molecules of the target nucleic acid. In some embodiments, the number of occurrences of the molecules of the target nucleic acid is, is indicated by, or is determined using, a number of the barcoded nucleic acids comprising a sequence of the target nucleic acid and different molecular barcode sequences in the sequences of the barcoded nucleic acids.

For example, the profile can include an RNA expression profile and/or a protein expression profile. The expression profile can comprise an RNA expression profile, an mRNA expression profile, and/or a protein expression profile. A profile can also be a profile of one or more target nucleic acids (e.g. gene markers) or a selection of genes associated with the cell.

In some embodiments, the method can be used to determine a profile (e.g., an expression profile, an omics profile, or a multi-omics profile) of a cell before or after the cells are subject to a pharmaceutical agent, such as to detect changes in gene expression profile of the cell in terms of identification of RNA transcripts and their quantitation. In some embodiments, a profile of a cell before or after being subject to a pharmaceutical agent can be determined using the nucleic acid sequences of the barcoded nucleic acids. For example, determining the profile of a cell before or after being subject to a pharmaceutical agent can comprise determining the profile of the cell using the molecular barcode sequences (e.g., UMI) and sequences of the target nucleic acids, or a portion thereof, present in the nucleic acid sequences.

In some embodiments, the cell before being subject to the pharmaceutical agent can have an expression profile different from an expression profile of the cell after being subject to the pharmaceutical agent. A differential expression analysis can be performed to detect quantitative changes in expression levels of the cell after being subject to the pharmaceutical agent. Genes expressed differentially can be detected. Differential expression profile can be correlated to the cell's functionality and/or cell's phenotypes, such as cell viability, cell activity, cell size, cell morphology, protein expression level, and/or signaling behaviors in response to the pharmaceutical agent.

Therefore, in some embodiments, efficacy of specific pharmaceutical agents and/or or specific conditions (such as concentration, temperature, duration) can be screened in a high-throughput manner by the present method. Subjecting the cell to a specific pharmaceutical agent can result in a specific change in a profile of the cell. The profile can comprise a multi-omics profile, such as a genomics profile, a proteomics profile, a transcriptomics profile, an epigenomics profile, a metabolomics profile, a chromatics profile, or a combination thereof.

### Diseases

The methods, compositions, reagents, systems and kits disclosed herein can be used to select appropriate agents for treating a disease or disorder. The treatment can include prophylactic treatment (or preventive treatment), which reduces a likelihood of developing the disease or disorder, and therapeutic treatment, which relieves, to some extent, one or more of symptoms of the disease or disorder.

Agents subject to screening using the methods, compositions, reagents, systems and kits disclosed herein can be agents for treating diseases and disorders including but not limited to rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthropathy, arthropathy, Reiter's disease, psoriatic arthropathy, ulcerative colitic arthropathy, enteropathic synovitis, Chlamydia, Yersinia and Salmonella associated arthropathy, spondyloarthropathy, atheromatous disease/arteriosclerosis, atopic allergy, autoimmune bullous disease, Pemphigus vulgaris, Pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, acquired immunodeficiency disease syndrome, acquired immunodeficiency related diseases, hepatitis B, hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylo sing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, cholestasis, idiosyncratic liver disease, drug-induced hepatitis, Non-alcoholic Steatohepatitis, allergy and asthma, group B streptococci (GBS) infection, mental disorders, depression, schizophrenia, Th2 Type and Th1 Type mediated diseases, acute pain, chronic pain, cancer, lung cancer, breast cancer, stomach cancer, bladder cancer, colon cancer, pancreatic cancer, ovarian cancer, prostate cancer, rectal cancer, hematopoietic malignancies, leukemia, lymphoma, abetalipoprotemia, acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas (e.g., prostate, small intestine, endometrium, cervical canal, large intestine, lung, pancreas, gullet, rectum, uterus, stomach, mammary gland, and ovary), aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aortic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CIVIL), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic aterio sclerotic disease, diffuse Lewy body disease, dilated congestive cardiomyopathy, disorders of the basal ganglia, Down's syndrome in middle age, drug-induced movement disorders which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis A, H is bundle arrythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, asthenia, infantile spinal muscular atrophy, inflammation of the aorta, influenza A, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphedema, malaria, malignant lymphoma, malignant histiocytosis, malignant melanoma, meningitis, meningococcemia, metabolic/idiopathic, migraine headache, mitochondrial multi. system disorder, mixed connective tissue disease, monoclonal gammopathy, multiple myeloma, multiple systems degenerations, Mencel Dejerine-Thomas Shi-Drager degeneration, Machado-Joseph degeneration, myasthenia gravis, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral arteriosclerotic disease, peripheral vascular disorders, peritonitis, pernicious anemia, Pneumocystis carinii pneumonia, pneumonia, POEMS syndrome, polyneuropathy, organomegaly, endocrinopathy, monoclonal gammopathy, skin changes syndrome, post perfusion syndrome, post pump syndrome, post-MI cardiotomy syndrome, preeclampsia, progressive supranuclear palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, senile dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, Syncope, syphilis of the cardiovascular system, systemic anaphylaxis, systemic inflammatory response syndrome, systemic onset juvenile rheumatoid arthritis, T-cell or FAB ALL, telangiectasia, thromboangiitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, viral encephalitis/aseptic meningitis, viral-associated hemophagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, alopecia greata, anaphylaxis, anti-phospholipid antibody syndrome, aplastic anemia, arteriosclerosis, atopic eczema, atopic dermatitis, autoimmune dermatitis, autoimmune disorder associated with Streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (cis) with risk for multiple sclerosis, conjunctivitis, childhood onset psychiatric disorder, chronic obstructive pulmonary disease (COPD), dacryocystitis, dermatomyositis, diabetic retinopathy, diabetes mellitus, disk herniation, disk prolapse, drug induced immune hemolytic anemia, endocarditis, endometriosis, endophthalmitis, episcleritis, erythema multiforme, erythema multiforme major, gestational pemphigoid, Guillain-Barré syndrome (GBS), hay fever, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratoconjunctivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, pauciarticular JRA, peripheral artery occlusive disease (PAOD), peripheral vascular disease (PVD), peripheral artery, disease (PAD), phlebitis, polyarteritis nodosa, periarteritis nodosa, polychondritis, polymyalgia rheumatica, poliosis, polyarticular JRA, polyendocrine deficiency syndrome, polymyositis, polymyalgia rheumatica (PMR), post-pump syndrome, primary Parkinsonism, prostatitis, pure red cell aplasia, primary adrenal insufficiency, recurrent neuromyelitis optica, restenosis, rheumatic heart disease, sapho (synovitis, acne, pustulosis, hyperostosis, and osteitis), scleroderma, secondary amyloidosis, shock lung, scleritis, sciatica, secondary adrenal insufficiency, silicone associated connective tissue disease, sneddon-wilkinson dermatosis, spondilitis ankylosans, Stevens-Johnson syndrome (SJS), systemic inflammatory response syndrome, temporal arteritis, toxoplasmic retinitis, toxic epidermal necrolysis, transverse myelitis, TRAPS (tumor necrosis factor receptor, type 1 allergic reaction, type II diabetes, urticaria, usual interstitial pneumonia (UIP), vasculitis, vernal conjunctivitis, viral retinitis, Vogt-Koyanagi-Harada syndrome (VKH syndrome), wet macular degeneration, wound healing, Yersinia, and Salmonella associated arthropathy.

The disease can be an infectious disease. As used herein, the term “infectious diseases” refers to diseases caused by any pathogen or agent that infects mammalian cells, preferably human cells and causes a disease condition, such as, for example, bacteria, yeast, fungi, protozoans, Mycoplasma, viruses, prions, and parasites. Non-limiting examples of infectious diseases include (a) viral diseases such as diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenza virus), a paramyxovirus (e.g., parainfluenza virus, mumps virus, measles vims, and respiratory syncytial virus (RSV)), a coronavirus (e.g., SARS, SARS-Cov-2), a papovavirus (e.g., papillomaviruses, such as those that cause genital warts, common warts, or plantar warts), a hepadnavirus (e.g., hepatitis B vims), a flavivirus (e.g., hepatitis C virus or Dengue virus), or a retrovirus (e.g., a lentivirus such as HIV); (b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; (c) other infectious diseases, such Chlamydia, fungal diseases including but not limited to candidiasis, aspergillosis, histoplasmosis, cryptococcal meningitis, parasitic diseases including but not limited to malaria, Pneumocystis carnii pneumonia, leishmaniasis, cryptosporidiosis, toxoplasmosis, and trypanosome infection and prions that cause human disease such as Creutzfeldt-Jakob Disease (CJD), variant Creutzfeldt-Jakob Disease (vCJD), Gerstmann-Straussler-Scheinker syndrome, Fatal Familial Insomnia and kuru.

In some embodiments, the disease or disorder is associated with expression of a tumor antigen, including but not limited to, a proliferative disease, a precancerous condition, a cancer, a non-cancer related indication associated with expression of the tumor antigen, or a combination thereof.

In some embodiments, the disease or disorder is a blood disease, an immune disease, a neurological disease or disorder, a cancer, a solid tumor, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

In some embodiments, the disease or disorder is a cancer selected from: colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer selected from chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. The cancer is, in some embodiments, solid tumor cancer.

The disease or disorder can comprise, for example, a cancer, a cardiovascular disease (e.g., arrhythmias, aorta disease, atherosclerosis, cardiomyopathy, congenital heart disease, coronary artery disease, heart attack, heart failure, hypertension, or stroke), diabetes (e.g., Type 1, Type 2, or gestational diabetes), an infectious disease (e.g., one caused by bacteria, viruses, fungi, or parasites), a neurodegenerative disease (e.g., Alzheimer's disease, amyotrophic lateral sclerosis, multiple sclerosis, Huntington's disease, or Parkinson's disease) or a respiratory disease (e.g., asthma, chronic obstructive pulmonary disease (COPD), pulmonary fibrosis, cystic fibrosis, or pneumonia), or a combination thereof.

### Cells

The cells can be obtained from any organism of interest. A cell can be a mammalian cell, and particularly a human cell such as T cells, B cells, natural killer cells, stem cells, cancer cells, or any cells the functionality of which can be affected by the presence of a pharmaceutical agent or other cells (e.g. cells involved in cell-cell interaction).

Cells described herein can be obtained from, derived from, cultured from, or progenies of cells cultured from a cell sample. A cell sample comprising cells can be obtained from any source including a clinical sample and a derivative thereof, a biological sample and a derivative thereof, a forensic sample and a derivative thereof, and a combination thereof. A cell sample can be collected from any bodily fluids including, but not limited to, blood, urine, serum, lymph, saliva, anal, and vaginal secretions, perspiration and semen of any organism. A cell sample can be products of experimental manipulation including purification, cell culturation, cell isolation, cell separation, cell quantification, sample dilution, or any other cell sample processing approaches. A cell sample can be obtained by dissociation of any biopsy tissues of any organism including, but not limited to, skin, bone, hair, brain, liver, heart, kidney, spleen, pancreas, stomach, intestine, bladder, lung, esophagus.

In some embodiments, the cell sample is a clinical sample or a derivative thereof, a biological sample or a derivative thereof, an environmental sample or a derivative thereof, a forensic sample or a derivative thereof, or a combination thereof. In some embodiments, the cell sample is collected from blood, urine, serum, lymph, saliva, anal, and vaginal secretions, perspiration, and/or semen of any organism. In some embodiments, the cell sample is obtained from skin, bone, hair, brain, liver, heart, kidney, spleen, pancreas, stomach, intestine, bladder, lung, and/or esophagus of any organism.

In some embodiments, the cells are cultured cells, such as cells from a cultured cell line. In some embodiments, the cells comprise immune cells, fibroblast cells, stem cells, or cancer cells.

In some embodiments, the cells are obtained from, cultured from, or progenies of cells cultured from a cell sample of a disease or disorder disclosed herein. In some embodiments, the cells are suitable for studying, or testing pharmaceutical agents of treating, a disease or disorder disclosed herein.

In some embodiments, the cells are cancer cells, such as cells of a cancer described herein. Examples of cancer cells include, but are not limited to, bladder cancer cells (e.g., CRL-1472, CRL-1473, CRL-1749, CRL-2169, HTB-2, HTB-4, HTB-5, HTB-9), breast cancer cells (e.g., MCF-7, CRL-1897, CRL-1902, CRL-2983, CRL-2988, CRL-3127, CRL-3166, CRL-1897, CRL-3180), colon cancer cells (e.g., CCL-229, CCL-233, CCL-235, CCL-237, CCL-248, CCL-255, CRL-5792, HTB-37, HTB-39), endometrial cancer cells (e.g., CRL-1671), gastric cancer cells (e.g., CRL-1739, CRL-5822, CRL-5971, CRL-5973, CRL-5974, HTB-103, MKN-28, SNU638), leukemia cells (e.g., CCL-119, CCL-240, CCL-243, CRL-1582, CRL-1873, CRL-2724, TIB-202), liver cancer cells (e.g., CRL-2234, CRL-2236, CRL-2237, CRL-2238, CRL-8024, CRL-10741, HTB-52, HB-8065), lung cancer cells (e.g., CCL-256, CCL-257, CRL-5803, CRL-5872, CRL-5875, CRL-5877, CRL-5908, HTB-183), small cell lung cancer cells (CRL-11350), non-small cell lung cancer cells (e.g., A549, CRL-5803, CRL-5893, CRL-5908, CRL-9609, HTB-178), kidney cancer cells (e.g., CRL-7569, CRL-7629, HTB-46, HTB-47) ovarian (e.g., SKOV3, CRL-1572, HTB-75, HTB-78), pancreatic cancer cells (e.g., CRL-1682, CRL-1687, CRL-1918, CRL-1997, CRL-2172, CRL-2547, HTB-79, HTB-80), prostate cancer cells (e.g., CRL-1740, CRL-3031, CRL-3033, CRL-3314, CRL-3315, CRL-3470, HTB-81), and skin cancer cells (e.g., A-375, HTB-66, HTB-69, HTB-71, CRL-7724). In some embodiments, the cells comprise non-small cell lung cancer cells, such as A549.

In some embodiments, the cells comprise cells suitable for studying, or testing pharmaceutical agents of treating, a cardiovascular disease (e.g., CRL-1395, CRL-1444, CRL-1476, CRL-1730, CRL-1999, CRL-2018, and CRL-2581), diabetes (e.g., CRL-3237, CRL-3242, CRL-11506, PCS-210-010), an infectious disease (e.g. CCL-86, CCL-156, CCL-214), a neurodegenerative disease (e.g., ACS-5001, ACS-1013, CRL-2541, HTB-11), or a respiratory disease (e.g., PCS-301-011, PCS-301-013, CRL-1848, CRL-4051, CRL-9609).

Also disclosed herein include kits for pharmaceutical screening and kits for screening pharmaceutical agents. The kit for screening pharmaceutical agents can, for example, comprise a plurality of barcode molecules. The kit can comprise a microwell array comprising at least 100 microwells (or a multiwell plate or microfluidic device or cartridge comprising at least 100 wells). The kit can comprise instructions for screening pharmaceutical agents according to the method as described herein. For example, the kit can comprise particles (e.g., beads), each of which can comprise a plurality of the barcode molecules. The kit can further comprise one or more reagents for use in the present method. For example, the kit can further comprise a cell lysis agent, one or more enzymes (such as reverse transcriptase, polymerase), or a chemical reagent. The kit can further comprise cells disclosed herein for use in the present method. For example, the kit can comprise, comprise about, comprise at least, comprise at least about, comprise at most, or comprise at most about, 1000, 2000, 3000, 4000, 5000, 6000, 7000, 8000, 9000, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 5×10⁶, 1×10⁷, 5×10⁷, 1×10⁸, 5×10⁸, 1×10⁹, 5×10⁹, or 1×10¹⁰ cells.

### EXAMPLES

Some aspects of the embodiments discussed above are disclosed in further detail in the following example, which are not in any way intended to limit the scope of the present disclosure.

### Example 1

### mRNA Analysis for Drug-Treated Cancer Cells

To simultaneously implement RNA-Seq under multiple drug treatment conditions, barcode molecules (or barcoding tags) are used to capture and distinguish mRNAs from samples treated with different drugs.

In this Example, barcoding oligo-dT molecules were used to tag multiple samples through mRNA captured. The barcoding oligo-dT molecules comprise a PCR handle sequence, a well position specific barcode (or a partition barcode sequence), a random DNA sequence (or a molecular barcode sequence) as unique molecular index (UMI), and an oligo(dT) primer sequence. The PCR handle sequence acts as priming site for RT reactions and PCR amplification reactions. The well position specific barcode is used to label different samples in different wells (e.g., cells treated with different drugs). The UMI can be used to detect and quantify unique mRNA transcripts. After reverse transcribing the mRNA, RT enzyme adds oligo(dC) to the end of first-strand cDNA, which allows the template switching oligo (TSO) to bind. Samples were pooled after a one step RT-PCR. After pre-amplification and tagmentation, paired end libraries were sequenced (FIGS. 1A-1D).

GEXSCOPE® Single Cell RNA-seq Library Construction kit (Singleron Biotechnologies) was used to demonstrate the technical feasibility and the utility of the present method in massively parallel multiplex chemical transcriptomics. The experiment was conducted according to manufacturer's instructions with modifications described below.

A one step RT-PCR reaction was carried out to simplify RT and PCR amplification in a one-step reaction. The enzymes used for the reaction included Reverse Transcriptases and Taq DNA Polymerase. After reverse transcription, reactions (42° C., 90 min) are heated up to 95° C. for 5 min to activate Taq DNA Polymerase and inactivate the reverse transcriptase at the same time. The reaction system and procedures are shown in Table 1:

TABLE 1
Reaction conditions

| Step | Procedure | Cycle |
|---|---|---|
| 1 | 42° C. 90 min | — |
| 2 | 95° C. 5 min | — |
| 3 | 98° C. 20 s | |
| 4 | 65° C. 45 s | 4 |
| 5 | 72° C. 3 min | |
| 6 | 98° C. 20 s | |
| 7 | 67° C. 20 s | 12 |
| 8 | 72° C. 3 min | |
| 9 | 72° C. 5 min | — |
| 10 | 12° C. ∞ | — |

A lung cancer cell line (A549) was plated in a 96-well plate. Each well was treated with a drug or DMSO (solvent) for 24 hours. After the drug treatment, the cells were lysed by adding a cell lysis agent to the wells. The lysed cells were transferred to a 96-well PCR plate. mRNA molecules were barcoded according to GEXSCOPE® instructions using barcode molecules. The barcode molecules having a poly-dT sequence (e.g., barcoding oligo(dT)) hybridizes to the mRNA to label each treatment and mRNA transcripts. A one step RT-PCR amplification reaction was performed according to above procedure. The amplified cDNA was pooled together. After purification, part of the cDNA is used to construct a transcriptome sequencing library. The resulting RNA-seq library was sequenced on an Illumina Nova-Seq with PE150 mode and analyzed with CeleScope bioinformatics workflow (Singleron Biotechnologies), as shown in FIGS. 1C and 1D. The effectiveness of drugs based on differences in gene expression and signal pathways was evaluated.

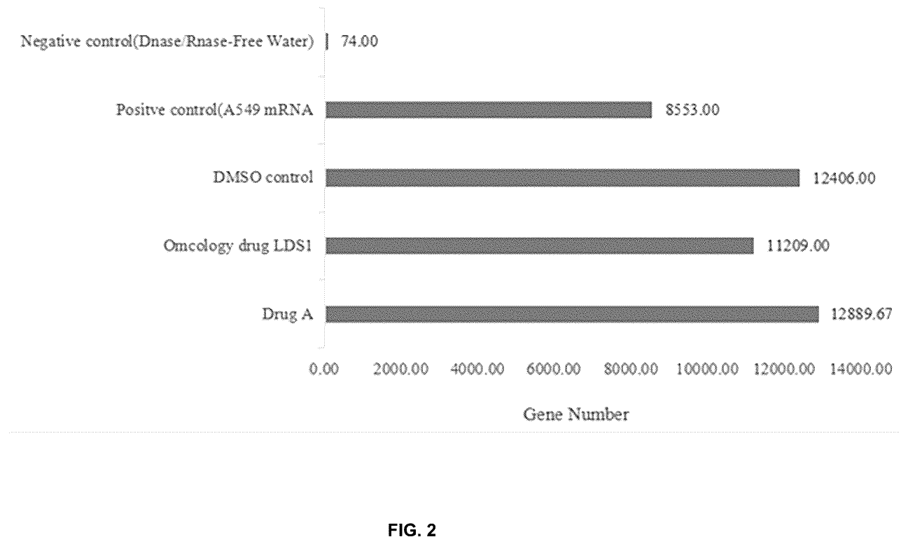

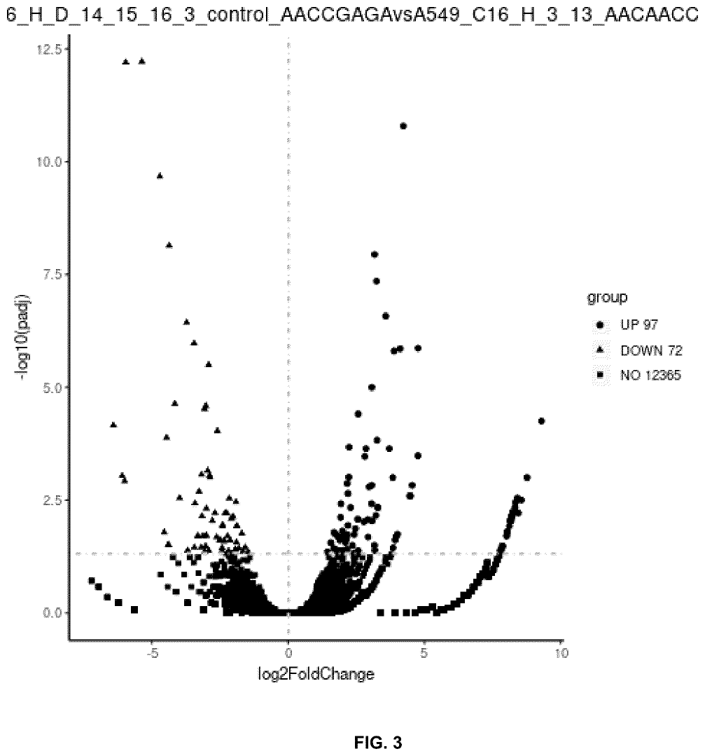

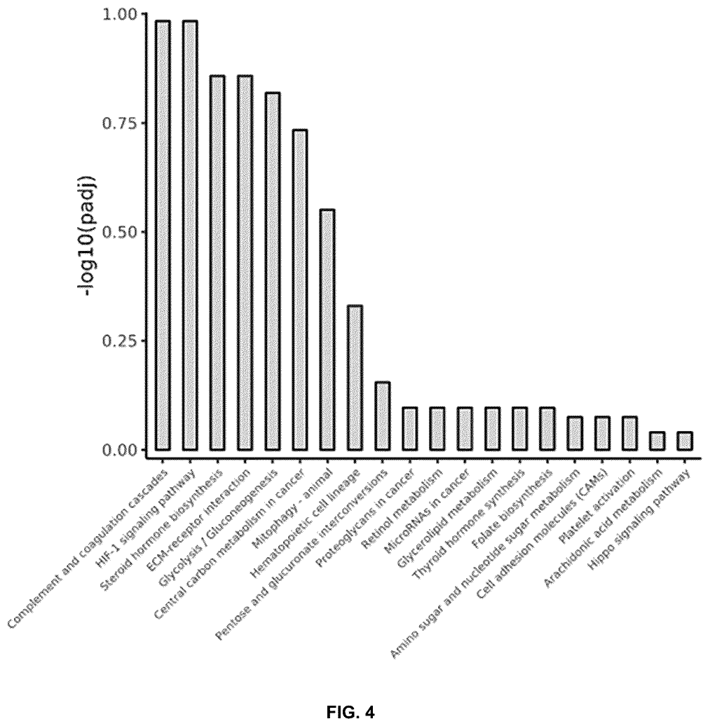

FIG. 2 shows the numbers of detected genes of in A549 tumor cells treated with different drugs (11209.00 and 12889.67) compared to cells treated with DMSO control (12406.00) and positive control (A549 mRNA, 8553.00). Results from differential expression analysis are shown FIG. 3. Results from pathway enrichment analysis are shown in FIG. 4.

### Example 2

### mRNA Analysis for Drug-Treated Cancer Cells

A549 (human non-small cell lung cancer cells) cells were plated in a 96-well plate for culture (1.0×10⁴ cells per well). The cells were treated either with drug solvent DMSO for 24 h or with a LSD1 inhibitor (10 μM) for 24 h. Barcoding molecules (oligo-dT) were used to barcode the mRNA molecules of the cells after treatment, using the one Step RT-PCR system as described in Example 1. Four replicates were prepared in each group. The product molecules in each well were purified and built separately.

Sequencing quality control indicators are shown in Table 2.

TABLE 2
Sequencing quality control indicators

| | Raw | Valid | Unique | Q30 of | Q30 of |
|---|---|---|---|---|---|
| Sample | Reads | Reads | Map | Barcodes | UMIs |
| DMSO | 10,810,036 | 10,309,382 | 8,956,476 | 95.90% | 92.99% |
| | | (95.37%) | (89.55%) | | |
| LSD1 | 9,664,404 | 9,331,654 | 7,945,390 | 95.27 | 93.54% |
| | | (96.56%) | (88.05%) | | |

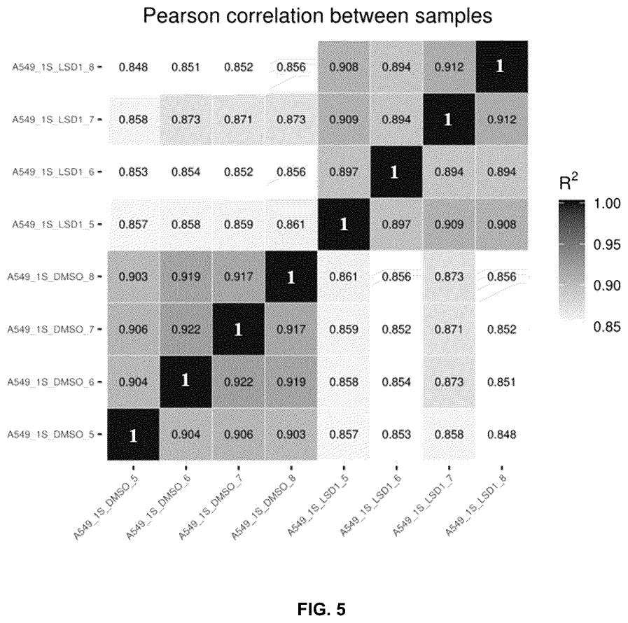

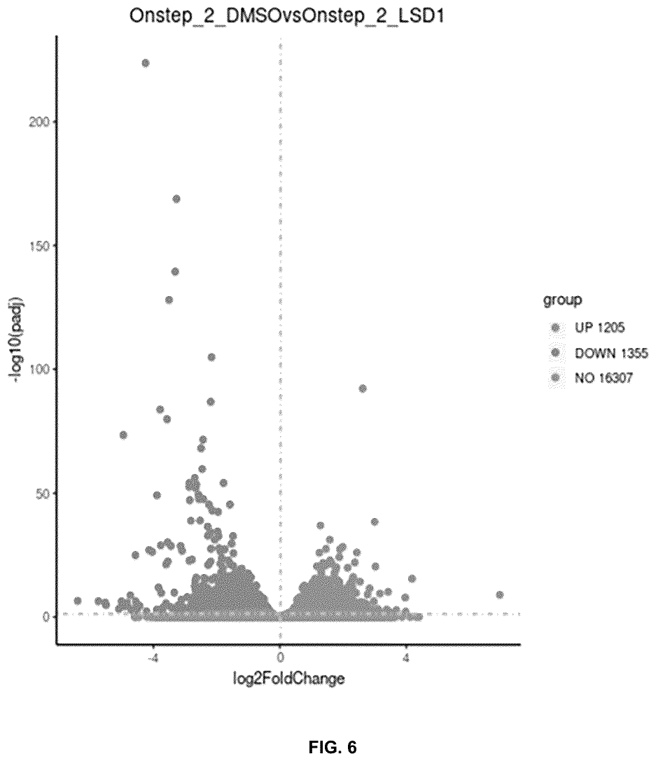

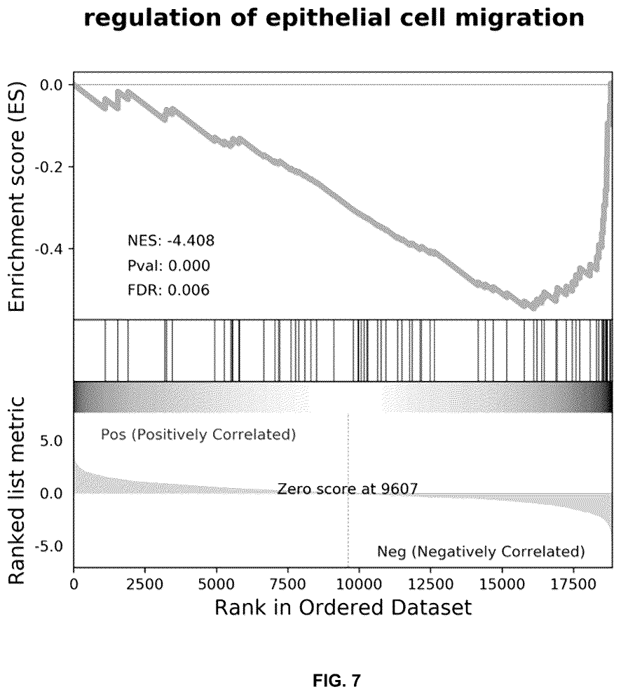

Results from parallel correlation analysis are shown FIG. 5. Results from differential gene analysis of the LSD1 inhibitor and the control are shown in FIG. 6. Results from enrichment (GO path) analysis of regulation of epithelial cell migration are shown in FIG. 7.

### Example 3

### One Step RT-PCR and Two Step Process Correlation Verification

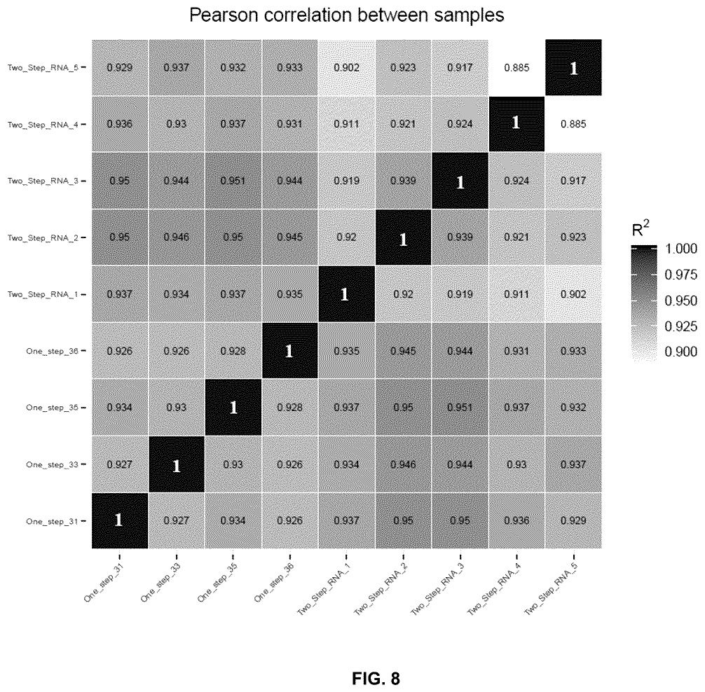

Using A549 cell line RNA as template (10 ng), reverse transcription and amplification were performed using a one-step method (as described in Examples 1 and 2) or a two-step method. The barcoded nucleic acids were subject to high-throughput sequencing for correlation analysis. It was found that the numbers of genes detected by the one step method and by the general two step method, at the same level of sequencing depth, were relatively consistent (Table 3), and the correlation between repetitions, including between groups, remained very high (parallel correlation analysis shown FIG. 8), indicating that the present one step process is highly efficient and accurate.

TABLE 3
Number of genes detected

| Sample Name | Gene Number | Unique Mapping |
|---|---|---|
| One_Step_31 | 16802 | 7072864(84.02%) |
| One_Step_33 | 15819 | 4783721(80.72%) |
| One_Step_35 | 16946 | 6710988(80.05%) |
| One_Step_36 | 15804 | 4620774(77.88%) |
| Two_Step_RNA_1 | 13942 | 3879146(88.47%) |
| Two_Step_RNA_2 | 15051 | 5102310(88.28%) |
| Two_Step_RNA_3 | 14909 | 5483066(87.71%) |
| Two_Step_RNA_4 | 14013 | 5459672(86.36%) |
| Two_Step_RNA_5 | 14785 | 4517305(88.59%) |

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of pharmaceutical screening, comprising:
introducing one or more cells into each partition of a plurality of partitions;
subjecting the one or more cells in the partition to a pharmaceutical condition of a plurality of pharmaceutical conditions;
introducing a plurality of barcode molecules into the partition, wherein the barcode molecules each comprises a partition barcode sequence and a molecular barcode sequence;
barcoding a plurality of target nucleic acids associated with the one or more cells in the partition using the plurality of the barcode molecules to generate a plurality of barcoded nucleic acids;
subjecting the plurality of barcoded nucleic acids, or products thereof, to sequencing to obtain sequences of the barcoded nucleic acids;
determining a profile of the one or more cells from the sequences of the barcoded nucleic acids; and
determining a difference in the profile of the cells subjected to two different pharmaceutical conditions of the plurality of conditions.

2. A method of pharmaceutical screening, comprising:
providing a plurality of partitions each comprising one or more cells, or lysates thereof, subjected to a pharmaceutical condition of a plurality of pharmaceutical conditions;
barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence;
subjecting the plurality of barcoded nucleic acids, or products thereof, to sequencing to obtain sequences of the barcoded nucleic acids;
determining a profile of the one or more cells from the sequences of the barcoded nucleic acids; and
determining a difference in the profile of the cells subjected to two different pharmaceutical conditions of the plurality of conditions.

3. A method of pharmaceutical screening, comprising:
providing a plurality of partitions each with a corresponding pharmaceutical condition of a plurality of pharmaceutical conditions, wherein partitions of the plurality of partitions each comprises one or more cells, or lysates thereof, subjected to the corresponding pharmaceutical condition of the partition;
barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence;
subjecting the plurality of barcoded nucleic acids, or products thereof, to sequencing to obtain sequences of the barcoded nucleic acids;
determining a profile of the one or more cells from the sequences of the barcoded nucleic acids; and
determining a difference in the profile of the cells subjected to two different pharmaceutical conditions of the plurality of conditions.

4. The method of any one of claims 1-3, wherein the plurality of pharmaceutical conditions comprise one or more pharmaceutical agents each at one or more concentrations and/or one or more control conditions, optionally wherein the one or more control conditions comprise a solvent control, a negative control, and/or a positive control.

5. A method of screening pharmaceutical agents, comprising:
introducing one or more cells into each partition of a plurality of partitions;
subjecting the one or more cells in the partition to a pharmaceutical agent of a plurality of pharmaceutical agents;
introducing a plurality of barcode molecules into the partition, wherein the barcode molecules each comprises a partition barcode sequence and a molecular barcode sequence;
barcoding a plurality of target nucleic acids associated with the one or more cells in the partition using the plurality of the barcode molecules to generate a plurality of barcoded nucleic acids;
subjecting the plurality of barcoded nucleic acids, or products thereof, to sequencing to obtain sequences of the barcoded nucleic acids;
determining a profile of the one or more cells from the sequences of the barcoded nucleic acids; and
analyzing the profile to determine an effect of the pharmaceutical agent on the one or more cells.

6. A method of screening pharmaceutical agents, comprising:
introducing one or more cells into each partition of a plurality of partitions;
subjecting the one or more cells in the partition to a pharmaceutical agent of a plurality of pharmaceutical agents;
introducing a plurality of barcode molecules into the partition, wherein the barcode molecules each comprises a partition barcode sequence and a molecular barcode sequence;
barcoding a plurality of target nucleic acids associated with the one or more cells in the partition using the plurality of the barcode molecules to generate a plurality of barcoded nucleic acids;
subjecting the plurality of barcoded nucleic acids, or products thereof, to sequencing to obtain sequences of the barcoded nucleic acids; and
analyzing the sequences of the barcoded nucleic acids to determine an effect of the pharmaceutical agent on the one or more cells.

7. A method of screening pharmaceutical agents, comprising:
introducing one or more cells into each partition of a plurality of partitions;
subjecting the one or more cells in the partition to a pharmaceutical agent of a plurality of pharmaceutical agents;
introducing a plurality of barcode molecules into the partition, wherein the barcode molecules each comprises a partition barcode sequence and a molecular barcode sequence;
barcoding a plurality of target nucleic acids associated with the one or more cells in the partition using the plurality of the barcode molecules to generate a plurality of barcoded nucleic acids; and
analyzing the plurality of barcoded nucleic acids to determine an effect of the pharmaceutical agent on the one or more cells.

8. A method of screening pharmaceutical agents, comprising:
introducing one or more cells into each partition of a plurality of partitions;
subjecting the one or more cells in the partition to a pharmaceutical agent of a plurality of pharmaceutical agents;
barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence; and
analyzing the plurality of barcoded nucleic acids to determine an effect of the pharmaceutical agent on the one or more cells.

9. A method of screening pharmaceutical agents, comprising:
providing a plurality of partitions each comprising one or more cells subjected to a pharmaceutical agent of a plurality of pharmaceutical agents;
barcoding a plurality of target nucleic acids associated with the one or more cells in the partition using a plurality of the barcode molecules to generate a plurality of barcoded nucleic acids, wherein the barcode molecules each comprises a partition barcode sequence and a molecular barcode sequence; and
analyzing the plurality of barcoded nucleic acids to determine an effect of the pharmaceutical agent on the one or more cells.

10. The method of any one of claims 7-9, wherein analyzing the plurality of barcoded nucleic acids comprises analyzing the sequences of the barcoded nucleic acids to determine an effect of the pharmaceutical agent on the one or more cells.

11. The method of any one of claims 6-10, wherein analyzing the sequences of the barcoded nucleic acids comprises:
determining a profile of the one or more cells from the sequences of the barcoded nucleic acids; and
analyzing the profile to determine an effect of the pharmaceutical agent on the one or more cells.

12. The method of any one of claims 5-11, wherein analyzing the profile comprises determining a difference between the profile and another profile, the difference being the effect of the pharmaceutical agent on the one or more cells, optionally wherein the method comprises receiving the other profile or the other profile is the profile of the one or more cells in another partition of the plurality of partitions whose associated plurality of target nucleic acids is barcoded to generate a plurality of barcoded nucleic acids which is analyzed to determine the other profile, optionally wherein the profile and the other profile are determined from similar numbers of cells.

13. The method of any one of claims 1-12, further comprising releasing the plurality of target nucleic acids associated with the one or more cells in the partition prior to barcoding the plurality of target nucleic acids.

14. The method of claim 13, wherein releasing the plurality of target nucleic acids associated with the one or more cells comprises lysing the plurality of cells.

15. The method of any one of claims 1-14, wherein the plurality of target nucleic acids comprise deoxyribonucleic acid (DNA), genomic DNA (gDNA), ribonucleic acid (RNA), and/or messenger RNA (mRNA).

16. The method of claim 1-15, wherein the barcode molecule further comprises a target binding sequence.

17. The method of claim 16, wherein the target binding sequence comprises a poly(dT) sequence and/or a sequence capable of hybridizing to the plurality of target nucleic acids.

18. The method of any one of claims 1-17, comprising introducing the plurality of the barcode molecules into the partition prior to subjecting the one or more cells in the partition with the pharmaceutical agent.

19. The method of any one of claims 1-18, comprising introducing the plurality of the barcode molecules into the partition after subjecting the one or more cells in the partition with the pharmaceutical agent.

20. The method of any one of 1-19, wherein barcoding the plurality of target nucleic acids comprises a reverse transcription reaction, and wherein the plurality of barcoded nucleic acids comprises complementary deoxyribonucleic acid (cDNA).

21. The method of any one of claims 1-20, wherein barcoding the plurality of target nucleic acids comprises extending the plurality of barcode molecules using the plurality of target nucleic acids as templates to generate the plurality of barcoded nucleic acids comprising a plurality of single-stranded barcoded nucleic acids, optionally hybridized to the plurality of target nucleic acids in the partition.

22. The method of claim 21, further comprising introducing a plurality of template switching oligonucleotides into the partition, wherein barcoding the plurality of target nucleic acids comprises extending the plurality of barcode molecules using the plurality of target nucleic acids and the plurality of template switching oligonucleotides as templates to generate the plurality of barcoded nucleic acids comprising a plurality of single-stranded barcoded nucleic acids.

23. The method of any one of claims 1-20, further comprising introducing a plurality of extension primers to the partition, and wherein barcoding the plurality of target nucleic acids comprises extending the plurality of extension primers using the plurality of target nucleic acids as templates and the plurality of barcode molecules as template switching oligonucleotides to generate the plurality of barcoded nucleic acids comprising a plurality of single-stranded barcoded nucleic acids.

24. The method of any one of claims 21-23, wherein each of the plurality of single-stranded barcoded nucleic acids is hybridized to one of the plurality of target nucleic acids and one of the plurality of template switching oligonucleotides in the partition.

25. The method of any one of claims 21-24, further comprising removing the plurality of target nucleic acids and the plurality of template switching oligonucleotides hybridized to the single-stranded barcoded nucleic acids, optionally wherein removing the plurality of target nucleic acids comprises denaturation, thermal denaturation, digesting, or hydrolyzing the plurality of target nucleic acids.

26. The method of any one of claims 1-24, wherein each of the plurality of single-stranded barcoded nucleic acid comprises a sequence of a barcode molecule of the plurality of barcode molecules, a sequence of a target nucleic acid of the plurality of target nucleic acids, a sequence of a template switching oligonucleotide of the plurality of template switching oligonucleotides, and/or a sequence of an extension primer of the plurality of extension primers.

27. The method of any one of claims 25-26, further comprising amplifying the plurality of barcoded nucleic acids to generate a plurality of double-stranded barcoded nucleic acids in the partition using the single-stranded barcoded nucleic acids as templates.

28. The method of claim 27, wherein amplifying the plurality of barcoded nucleic acids comprises amplifying the plurality of barcoded nucleic acids in the partition to generate the plurality of double-stranded barcoded nucleic acids, wherein the plurality of target nucleic acids in a partition are barcoded and the plurality of barcoded nucleic acids generated are then amplified in the same partition, and/or wherein the plurality of target nucleic acids in a partition are barcoded and the plurality of barcoded nucleic acids generated are then amplified in the same reaction.

29. The method of any one of claims 27-28, wherein each of the plurality of barcode molecules comprises a primer sequence, optionally wherein the primer sequence comprises a PCR primer sequence, wherein amplifying the plurality of barcoded nucleic acids comprises amplifying the plurality of barcoded nucleic acids using the primer sequences in single-stranded barcoded nucleic acids of the plurality of single-stranded barcoded nucleic acids, or products thereof.

30. The method of any one of claims 1-29, further comprising pooling the plurality of barcoded nucleic acids, or products thereof, in each of the plurality of partitions to generate pooled barcoded nucleic acids, wherein subjecting the plurality of barcoded nucleic acids, or products thereof, to sequencing comprises subjecting the pooled barcoded nucleic acids, or products thereof, to sequencing, optionally wherein pooling the plurality of barcoded nucleic acids, or products thereof, comprises pooling the plurality of double-stranded barcoded nucleic acids in each of the plurality of partitions to generate the pooled barcoded nucleic acids.

31. The method of any one of claims 1-30, further comprising fragmenting the pooled barcoded nucleic acids to generate fragmented barcoded nucleic acids to generate fragmented barcoded nucleic acids prior to subjecting the plurality of barcoded nucleic acids, or products thereof, to sequencing.

32. The method of claim 31, wherein fragmenting the pooled barcoded nucleic acids comprises enzymatic fragmentation, physical fragmentation, or a combination thereof.

33. The method of claim 32, wherein the enzymatic fragmentation comprises the use of one or more restriction enzymes.

34. The method of any one of claims 1-33, comprising performing a polymerase chain reaction in bulk on the pooled barcoded nucleic acids, or the fragmented barcoded nucleic acids, to generate amplified barcoded nucleic acids.

35. The method of claim 34, wherein performing the polymerase chain reaction in bulk is subsequent to fragmenting the pooled barcoded nucleic acids.

36. The method of any one of claims 34-35, wherein the amplified barcoded nucleic acids comprise a sequence for attaching the amplified barcoded nucleic acids to a flow well.

37. The method of claim 36, wherein the sequence for attaching the amplified barcoded nucleic acids to the flow well is a P5 sequence, a P7 sequence, or a portion thereof.

38. The method of any one of claims 34-37, wherein the amplified barcoded nucleic acids comprise a sequencing primer sequence.

39. The method of any one of claims 1-38, wherein the profile comprises a multi-omics profile, optionally wherein the multi-omics profile comprises a genomics profile, a proteomics profile, a transcriptomics profile, an epigenomics profile, a metabolomics profile, a chromatics profile, a protein expression profile, a cytokine secretion profile, or a combination thereof.

40. The method of any one of claims 1-39, wherein the profile comprises an expression of a target nucleic acid of the plurality of target nucleic acids, optionally wherein the expression of the target nucleic acid comprises an abundance of the target nucleic acid, optionally wherein the abundance of the target nucleic acid comprises an abundance of molecules of the target nucleic acid barcoded using the barcode molecules, optionally wherein the abundance of the molecules of the target nucleic acid comprises a number of occurrences of the molecules of the target nucleic acid, optionally wherein the number of occurrences of the molecules of the target nucleic acid is, is indicated by, or is determined using, a number of the barcoded nucleic acids comprising a sequence of the target nucleic acid and different molecular barcode sequences in the sequences of the barcoded nucleic acids.

41. The method of any one of claims 1-40, wherein at least two of the partition barcode sequences of the plurality of barcode molecules in the same partition are identical.

42. The method of any one of claims 1-41, wherein the partition barcode sequences of at least one barcode molecules in at least two different partitions are different.

43. The method of any one of claims 1-42, wherein at least two of the molecular barcode sequences of the plurality of barcode molecules in a partition comprise different molecular barcode sequences, optionally wherein the molecular barcode sequences are unique molecular identifier.

44. The method of any one of claims 1-43, wherein each of the plurality of barcode molecules comprises a primer sequence, optionally wherein the primer sequence is a sequencing primer sequence, optionally wherein the sequencing primer sequence is a Read 1 sequence, a Read 2 sequence, or a portion thereof.

45. The method of any one of claims 1-44, wherein the one or more cells comprise at least 10 cells, 100 cells, 1000 cells, or 10000 cells.

46. The method of any one of claims 1-45, wherein the one or more cells are obtained from, cultured from, or progenies of cells cultured from, a cell sample.

47. The method of claim 46, wherein the cell sample is a clinical sample or a derivative thereof, a biological sample or a derivative thereof, a forensic sample or a derivative thereof, or a combination thereof.

48. The method of any one of claims 46-47, wherein the cell sample is collected from blood, urine, serum, lymph, saliva, anal, and vaginal secretions, perspiration, and/or semen of any organism.

49. The method of any one of claims 46-48, wherein the cell sample is obtained from skin, bone, hair, brain, liver, heart, kidney, spleen, pancreas, stomach, intestine, bladder, lung, and/or esophagus of any organism.

50. The method of any one of claims 46-49, wherein the cells are cultured cells.

51. The method of any one of claims 46-50, wherein the cells comprise immune cells, fibroblast cells, stem cells, or cancer cells.

52. The method of any one of claims 1-51, wherein introducing the plurality of barcode molecules to the partition comprises introducing a particle comprising the plurality of barcode molecules to the partition.

53. The method of claim 52, wherein the plurality of barcode molecules are attached to, reversibly attached to, covalently attached to, or irreversibly attached to the particle.

54. The method of claim 53, wherein the particle is a gel particle, optionally wherein the gel bead is a hydrogel particle.

55. The method of claim 54, wherein the gel particle is degradable upon application of a stimulus.

56. The method of claim 55, wherein the stimulus comprises a thermal stimulus, a chemical stimulus, a biological stimulus, a photo-stimulus, or a combination thereof.

57. The method of claim 53, wherein the particle is a solid particle and/or a magnetic particle.

58. The method of claim 57, wherein the particle is retained in the partition by an external magnetic field during one or more steps of the method.

59. The method of claim 58, wherein the particle comprises a paramagnetic material.

60. The method of any one of claims 57-59, wherein the particle has a size of about 10 μm to about 100 μm.

61. The method of any one of claims 1-60, wherein the plurality of partitions comprise a plurality of microwells of a microwell array.

62. The method of claim 61, wherein the plurality of partitions comprises at least 100 partitions.

63. The method of any one of claims 1-62, wherein the pharmaceutical agent comprises one or more therapeutic compounds, one or more hormones, one or more antibodies, one or more therapeutic peptides, one or more therapeutic nucleic acids, or combinations thereof.

64. The method of claim 63, wherein the pharmaceutical agent comprises an anti-cancer compound.

65. The method of any one of claims 63-64, wherein the cells in two partitions are subject to two different pharmaceutical agents.

66. The method of any one of claims 63-65, wherein the cells in two partitions are subject to one pharmaceutical agent under different conditions.

67. The method of claim 66, wherein the different conditions comprise different concentration of the pharmaceutical agent, dosage regimen of the pharmaceutical agent, temperature, duration, presence of one or more additional agents, or a combination thereof.

68. The method of any one of claims 1-67, wherein the cells in the plurality of partitions are subject to at least 10 different pharmaceutical agents and/or one pharmaceutical agent under at least 10 different control conditions.

69. A kit for screening pharmaceutical agents, comprising:
a plurality of barcode molecules;
a microwell array comprising at least 100 microwells; and
instructions to use the kit for pharmaceutical screening or screening pharmaceutical agents according to the method of any one of claims 1-68.

70. The kit of claim 69, further comprising one or more reagents used in the method.
